SEQUENCE LISTING (13 sequences)

SEQ ID NO: 1 - protein, 15 aa, Human immunodeficiency virus type 1
MRQAHCNISRAKWNN

SEQ ID NO: 2 - protein, 15 aa, Human immunodeficiency virus type 1
MRQAHCNISRAKWNA

SEQ ID NO: 3 - protein, 15 aa, Human immunodeficiency virus type 1
IRQAHCNISRAKWND

SEQ ID NO: 4 - protein, 15 aa, Artificial Sequence
  Description of Artificial Sequence PEPTIDE
IRQAHCNVSRSEWNK

SEQ ID NO: 5 - protein, 15 aa, Artificial Sequence
  Description of Artificial Sequence PEPTIDE
IRQAHCNISRAQWNN

SEQ ID NO: 6 - protein, 14 aa, Homo sapiens
SKQVCADPSESWVQ

SEQ ID NO: 7 - protein, 14 aa, Homo sapiens
NRQVCIDPKLKWIQ

SEQ ID NO: 8 - protein, 15 aa, Artificial Sequence
  Description of Artificial Sequence PEPTIDE KK
IRKAHCNISRAKWND

SEQ ID NO: 9 - protein, 15 aa, Artificial Sequence
  Description of Artificial Sequence PEPTIDE 15D
IRKAHCNISRADWND

SEQ ID NO: 10 - protein, 15 aa, Artificial Sequence
  Description of Artificial Sequence PEPTIDE 15CW
IRKAHCWIDRADNNS

SEQ ID NO: 11 - protein, 15 aa, Artificial Sequence
  Description of Artificial Sequence PEPTIDE KS
KINSWRADNIHCKAR

SEQ ID NO: 12 - protein, 15 aa, Artificial Sequence
  Description of Artificial Sequence PEPTIDE 15GIG
GIGDPVTCLKSGAIA

SEQ ID NO: 13 - protein, 6 aa, Artificial Sequence
  Description of Artificial Sequence PUTATIVE HIV GP120 V3 CONSENSUS SEQUENCE
GPGRAF

(19) United States
(12) Patent Application Publication
(10) Pub. No.: US 2006/0116325 A1
(43) Pub. Date: Jun. 1, 2006

(54) Methods and compositions for inhibiting hiv-coreceptor interactions

(76) Inventors: Oleg Chertov, Frederick, MD (US); Joost J Oppenheim, Bethesda, MD (US); Xin Chen, Frederick, MD (US); Connor McGrath, Union Bridge, MD (US); Raymond C Sowder II, Frederick, MD (US); Jacek Lubkowski, Frederick, MD (US); Michele Wetzel, Souderton, PA (US); Thomas J Rogers, North Wales, PA (US)

Correspondence Address: TOWNSEND AND TOWNSEND AND CREW, LLP, TWO EMBARCADERO CENTER, 8TH FLOOR, SAN FRANCISCO, CA, 94111, US

(21) Appl. No.: 10/468,182

(22) PCT Filed: Feb. 15, 2002
(86) PCT No.: PCT/US02/05063

§ 371 Date: Aug. 20, 2004

Related U.S. Application Data
(60) Provisional application No. 60/269,534, filed on Feb. 15, 2001.

(51) Int. Cl. A61K 38/10

(52) U.S. Cl. 514/13; 514/14

(57) ABSTRACT

Novel methods and compositions are provided for inhibiting interactions between human immunodeficiency viruses (HIVs) and viral coreceptors, including CXCR4 and/or CCR5 coreceptors. The anti-coreceptor binding agent includes a novel peptide portion of the gp120 envelope protein of HIV-1, as well as peptide analogs and mimetics of this peptide, that specifically binds to, or modulates activity of, the coreceptors(s). The anti-coreceptor binding agent is useful as a prophylactic or therapeutic treatment to prevent or inhibit HIV binding to a susceptible cell and thereby reduces infection and/or moderates or treats related diseases. In alternative embodiments, the peptides, analogs and mimetics are effective to inhibit direct co-receptor binding by HIV virus, coreceptor binding by HIV gp120 proteins or peptides, HIV fusion with target host cells, HIV virion entry into host cells, HIV replication, and HIV transmission between cells and hosts. In more detailed embodiments, the anti-coreceptor binding agents of the invention are multi-tropic by exhibiting activity against HIV interactions with multiple, CXCR4 and CCR5, coreceptors.

### RELATED APPLICATION

This application claims priority to provisional application Ser. No. 60/269,534 filed on Feb. 15, 2001.

### BACKGROUND OF THE INVENTION

The envelope glycoprotein of the human immunodeficiency virus type I (HIV-1) mediates in the fusion of viral and host cell membranes necessary for virion entry (Freed et al., J. Biol. Chem. 270:23883-23886, 1995). The envelope glycoprotein of HIV-1 is produced by the enzymatic cleavage from a gp160 precursor protein to produce the external gp120 protein and the transmembrane gp41 protein (Capon et al., Annu. Rev. Immunol. 9:649-678, 1991).

Several studies have identified specific portions or domains of the gp120 protein that may elicit humoral and/or cell-mediated immune responses to HIV in susceptible host subjects, and may therefore be useful to formulate anti-HIV reagents and methods for prevention and treatment of HIV infection and related diseases. These general HIV peptide studies describe a large, diverse assemblage of gp120 peptides that are proposed as candidates for therapeutic use, primarily in vaccine formulations to prevent and treat HIV infection and related disease. For example, U.S. Pat. No. 5,691,135 describes various peptides that are selected for the ability to inhibit HIV infection by stimulating VH3 and VH4 antibody responses. The peptides are proposed for administration to a patient as an antigen in sufficient quantity to induce antibodies that exhibit superantigen binding to gp120. The disclosure considers 31 peptides obtained from the AIDS Research and Reference Program, NIH, which peptides correspond to sequences from gp120 and gp41 from different strains of HIV. Additional gp120 peptides are described in U.S. Pat. No. 5,939,074. In particular, this references describes peptides that are useful as “multideterminant peptide antigens” for eliciting both cell-mediated and humoral immune responses against HIV.

Additional studies have identified particular regions of the gp120 that are proposed to determine specific interactions between gp120 and CD4, which is the primary receptor on target cells mediating cellular entry by HIV-1 (Arthos et al., Cell 57:469481, 1989; Clayton et al., Nature 339:548-551, 1989; Kwong et al., Nature 393:648-659, 1998; and Landau et al., Nature 334:159-162, 1988).

In addition to CD4 binding, cellular entry by HIV-1 is thought to require additional interactions between the virus and one or more “coreceptors” on the surfaces of target cells. Briefly, the HIV-1 particles are proposed to bind initially to the CD4 receptor, and then subsequently to a chemokine receptor present on the target cells, which is used by the virus as a “coreceptor” for mediating cellular entry. Different strains of HIV-1 preferentially utilize different chemokine receptors that are variably expressed among HIV-1 target cells. In particular, some strains of HIV-1, referred to as “lymphotropic” strains, bind CXCR4 chemokine receptors predominantly expressed on lymphocyte target cells, while other HIV-1 strains, termed “monocyte tropic” strains, bind CCR5 receptors predominantly expressed on cells of monocyte lineage. Thus, the major viral coreceptors are CXCR4 (Berson et al., J. Virol. 70:6288-6295, 1996), which has a native function as a chemokine receptor for stromal derived factor-1 (SDF-1), and CCR5 (Alkhatib et al., Science 272:1955-1958, 1996; Deng et al., Nature 381:661-666, 1996; and Dragic et al., Nature 381:667-673, 1996), which functions naturally as a receptor for several chemokines, including macrophage inflammatory protein-1β (MIP-1β).

It is proposed that a conformational change occurs in gp120 following HIV binding of CD4, and that this conformation change exposes one or more binding sites on the gp120 molecule that mediate additional interactions between HIV and chemokine receptor, which are referred to in this context as “viral coreceptors” (Kwong et al., Nature 393:648-659, 1998, incorporated herein by reference). Recent studies suggest a complicated, multi-step binding and activation model, wherein the association of gp120 with CD4 yields a CD4-gp120 complex, which subsequently associates with the viral coreceptor resulting in a structural rearrangement (e.g., conformational change) of gp120 that facilitates interaction of the gp41 envelope protein subunit with the host cell membrane, leading to viral entry (Helseth et al., J. Virol. 64:6314-6318, 1990; and Weissenhom et al., Nature 387:426-430, 1997).

Within this emergent field of investigation, numerous studies have focused on structure-function mapping of chemokine receptors, with the goals of determining binding determinants and mechanisms of action of the receptors. Yet additional research has focused on chemokine mapping studies. From these studies chemokine-derived peptides have been identified that reportedly comprise binding determinants of the chemokines capable of blocking native chemokine receptor-ligand interactions. Further studies, focusing alternatively on HIV gp120 mapping, report production and testing of gp120 peptides capable of blocking HIV-coreceptor binding and HIV infectivity.

With regard to chemokine receptor studies, numerous articles report that chemokine receptors play a direct role as coreceptors for HIV cell entry. Briefly, as noted above, it is reported that specific interactions between the HIV envelope glycoprotein gp120 and one or more chemokine receptors mediate viral entry into target cells. More specifically, monocyte-tropic or “m-tropic” HIV strains bind to a distinct chemokine receptor, CCR5, for cell entry, while T cell lymphotropic or “T-tropic” virus use mainly CXCR4 receptors for cell entry. More detailed, structure-function analyses have been directed toward identifying specific HIV binding determinants of chemokine receptors mediating their activity as coreceptors for HIV entry.

In conjunction with these reports, certain references focus on a concept of blocking chemokine receptor activity (e.g., receptor binding with chemokines, or with gp120) using competitive inhibitors. Proposed inhibitors of chemokine receptor binding interactions include peptide inhibitors that mimic structures of chemokines, or of gp120, binding determinants or related structural domains. These studies follow more basic research which shows that intact chemokines, the normal ligands of chemokine receptors, can compete with cognate chemokines, or with HIV, for binding to the target receptors.

Referring specifically to chemokine mapping studies focusing on chemokine structure-function, various publications attempt to identify and characterize receptor binding determinants of chemokines. As noted above, certain of these references also describe chemokine-based peptides reportedly capable of blocking chemokine-receptor binding and other activities mediated by receptor-ligand (chemokine or HIV) interactions. One such study is presented in a publication by Reckless et al., (Biochem. J. 340:803-811, 1999). This study identifies a number of chemokine-derived peptides, including a peptide designated “peptide 3”, based on a human chemokine, monocyte chemotactic protein-1 (MCP-1). Reckless and colleagues report that the peptide 3 inhibits cell migration induced by a wide range of chemokines. Moreover, peptide 3 reportedly binds to THP-1 cells and inhibits THP-1 migration, reportedly by acting as a chemokine receptor antagonist. On this basis, the authors propose that peptide 3 and its derivatives, including peptides ranging from 6-15 residues in length, may be useful as chemokine inhibitors.

A number of related reports focus on a distinct portion of chemokines as prospective receptor binding determinants. In particular, the reports focus on the N-terminal region of chemokines comprising a structural element called the “N-loop”. This distinct element follows the first two cysteine residues of a model chemokine, and is proposed to play an important role in chemokine-receptor interactions. Other parts of chemokine molecules have also been proposed to contribute significantly as structural determinants of interactions between chemokines and their cognate receptors. For example, Crump et al., (EMBO J. 16:6996-7007, 1997), teach that the N-terminal eight residues of the chemokine SDF-1 form an important receptor binding site. At this site, two residues (Lys-1 and Pro-2) were proposed to be directly involved in receptor activation. Disruption of these residues reportedly abolished activation. It has further been reported that SDF-1 includes a second receptor binding motif at residues 12-17 of the chemokine loop region, termed the “RFFESH motif”.

Thus, it has been widely considered that the N-terminal region and so called N-loop following the first two cysteine residues of chemokines play the most important role in mediating the interactions between chemokines and their cognate receptors (Clark-Lewis et al., J. Biol. Chem. 266:23128-23134, 1991; Crump et al., EMBO J. 16:6996-7007, 1997; and Pakianathan et al., Biochemistry 36:9642-9648, 1997). In one of these studies, Clark-Lewis et al. reported that a C-terminally truncated form of IL-8 missing the α-helix and β-turn within this region manifested greatly reduced chemotactic activity. Additional confirmation of the primary significance of this segment of chemokines for receptor binding is provided by a recent observation that the biological activity of human MIP-1β is strongly reduced by substitutions of Arg-45, and Arg-47, with Serine (Czaplewski et al., J. Biol. Chem. 274:16077-16084, 1999, incorporated herein by reference). Finally, a peptide corresponding to the MCP-1 sequence just preceding the C-terminal α-helix was reported to inhibit chemotaxis of THP-1 cells indicating the importance of this region for chemokine function (Reckless et al., Biochem. J. 340:803-1121, 1999). Additional structure-function data for the CC chemokine RANTES have been reported by Pakianathan et al., (Biochemistry 36:9642-9648, 1997).

Notably, a number of the foregoing publications point to a complex, multi-determinant model of interactions between chemokines and their native receptors. In this context, Pakianathan et al. believe the data indicates that RANTES interacts with each of its receptors in a distinct and specific manner and supports a two-site model of interaction between chemokines and their receptors.

Protein mapping studies of HIV gp120 envelope have reported identification of structural determinants of gp120 responsible for mediating HIV-coreceptor interactions. Among these studies, Rizzuto et al., Science 280:1949-1953, 1998, have described a conserved gp120 structure that is reportedly important for binding to CCR5, and have postulated generally that this structural determinant should facilitate development of pharmacologic or immunologic inhibitors of virus-receptor interactions. Rizzuto et al., suggested “that the CCR5-binding site is likely composed of conserved gp120 elements near or within the bridging sheet and V3 loop residues.”

In another report addressing gp120 structure-function analysis relating to coreceptor binding, Verrier et al., (AIDS Res. Hum. Retroviruses 15:731-743, 1999), studied the effect of linear V3 peptides (21-30 amino acids in length) on infectivity of different strains of HIV-1. These studies also pointed to the V3 loop as an important determinant of coreceptor choice, whereby single amino acid substitutions in V3 were reported to dramatically alter coreceptor usage. In conjunction with this disclosure, Verrier et al., reported that artificial, linear peptides of V3 could compete with intact gp120 for binding to CCR5 and CXCR4 and block HIV entry into cells. More specifically, the authors reported that the most efficient peptides for blocking fusion were derived from the middle of V3, and therefore did not include sequences at the C-terminal or N-terminal ends of V3 that form the base of the V3 loop. Also significantly, Verrier et al., (supra) pointed to a “pattern of restriction” between multiple gp120 binding determinants, whereby peptides from different HIV strains (m-tropic versus lymphotropic) discriminate in their fusion-blocking activity in a pattern that “follows the coreceptor usage of the parental envelopes from which the peptides were derived.” This indicates that the candidate peptides described by Verrier et al., (supra) would not exhibit multi-specific blocking potential against both m-tropic and lymphotropic HIV-coreceptor interactions.

In a related study, Sakaida et al., (J. Virol. 72:9763-9670, 1998) reported that synthetic cyclized (but not linear) V3 peptides of CXCR4 and dual-tropic strains of HIV-1 (but not a CCR5 strain) can prevent binding of anti-CXCR4 antibodies, potentially by binding to the coreceptor and acting as a competitive inhibitor. These same peptides reportedly inhibited calcium mobilization by the chemokine SDF-1 in a T cell line. The reference proposes that V3 loop peptides can directly bind to the relevant chemokine receptor and determine coreceptor usage, and postulates that such peptides can serve as HIV-1 reagents. Like Verrier et al., (supra) Sakaida and coworkers conclude that the coreceptor binding activities of V3 peptides are strain-dependent.

More detailed reports pertaining to the role of V3 in mediating HIV-coreceptor interactions focus on specific sites or residues within the V3 domain as reportedly critical or important residues for coreceptor binding. For example, Tugarinov et al., (Structure Fold Des. 8:385-395, 2000), report that a conserved, central loop sequence, GPG, within the V3 loop plays a primary role in maintaining the conformation of the loop to mediate coreceptor binding. Kato et al. (J. Virol. 73:5520-5526, 1999) suggest that three specific residues, confined to the central, loop portion of V3 and located distant from the C-terminal segment, are particularly important for coreceptor usage. Xiao et al., (Virology 240:83-92, 1998), identify a consensus motif S/GXXXGPGXXXXXXXE/D, covering the central portion of the V3 loop and excluding the C-terminal portion, as a critical motif for coreceptor binding. Wang et al., Proc. Natl. Acad. Sci. USA 95:5740-5745, 1998), studied whether certain V3 residues conserved among HIV-1, HIV-2 and SIV determine the utilization of CCR5 as a coreceptor. They concluded that Arg-298 (at the beginning of V3 loop) has an importance for CCR5 utilization. The authors suggested that this residue may represent a highly conserved structural element and a useful target for developing anti-viral therapies.

In another study by Wang et al., (Proc. Natl. Acad. Sci. USA 96: 4558-4562, 1999), additional V3 residues reported to be critical for CCR5 utilization are identified by alanine scanning mutagenesis. One of the identified residues, A₃₂₈, is located at the C-terminus of V3, and its substitution reportedly results in a 1,000-fold reduction of CCR5 binding activity. However, this residue is only one of two “highly conserved” residues and six “critical” residues identified for CCR5 utilization. Most of these residues are distinctly located at the C-terminal or central loop portion of V3, including R₂₉₈, K₃₀₅, I₃₀₇, R₃₁₃, and F₃₁₅. Wang and colleagues propose that “these highly invariable residues” as well as others identified in a “bridging sheet” portion of the molecule may represent “targets for antiviral designs aimed at blocking the coreceptor entry step of HIV-1 replication.”

Based on the foregoing articles, there is a broadly-confirmed disclosure of V3 as a critical domain for mediating HIV-coreceptor interactions. A large number of important or critical residues are indicated, with a substantial range of discrepancy between reports implicating different residues as being important, or critical, for coreceptor binding functions. These references that focus on V3 uniformly teach away from excluding important “critical” or “highly conserved” V3 residues. At the same time, these references focusing on the V3 domain uniformly fail to implicate other sequence elements that may be present in the gp120 molecule outside the V3 loop portion thereof.

Coupled with these teachings, a host of related publications point to yet additional components of gp120 that may be essential for HIV-coreceptor interactions. Because these components appear to be complex and potentially interact to achieve coreceptor binding, their disclosure adds further complexity to understanding gp120 structure-function relationships for mediating coreceptor binding and cell entry. In particular, Tugarinov et al., (Structure Fold Des. 8:385-395, 2000), teach the importance of the GPG motif of V3, but also teach that “[h]igh affinity binding of gp120 to the chemokine receptors requires participation of other domains in gp120 such as the CD4i epitope.” Rizzuto et al., (Science 280:1949-1953, 1998), suggest that the “CCR5-binding site is likely composed of conserved gp120 elements near or within the bridging sheet and V3 loop residues.” They further proposed that CD4 binding may distort the V1/V2 stem, repositioning the stem allowing the formation of the β-sheet important for CCR5 binding. They also noted that substitution of Asp for Thr₁₂₃, located in the V1/V2 stem and which contact CD4, significantly decreased CCR5 binding.

This report parallels others which point to a favored model of a “conformational binding site” in gp120 for mediating HIV-coreceptor interactions. According to this model, effective binding of coreceptors by gp120 involves initial binding of the gp120 to a CD4 receptor, which brings about a “CD4 induced” conformational change in gp120 involving distant residues—that in turn leads to formation of a conformational binding determinant on gp120 to mediate HIV-coreceptor interactions. In this context, Wu et al., (Nature 384:179-183, 1996) suggested that HIV-1 attachment to CD4 creates a high-affinity binding site for CCR-5, leading to membrane fusion and virus entry.

Consistent with this model, recent studies have identified yet additional sites of gp120 which are reportedly important, or essential, for mediating HIV-coreceptor interactions. These sites are generally distinct from V3, and also from the V3-flanking regions. For example, Cho et al., (J. Virol. 72:2509-2515, 1998), report that V1/V2 and V3 confer on HIV-1 the ability to use CXCR4 coreceptors, but that the V4 and V5 regions are also “required in conjunction with regions V1 and V3 of HIV-1DH₁₂ gp120 for efficient utilization of CXCR4.” Este et al., (Mol. Pharmacol. 52:98-104, 1997), point to structural determinants in each of the V2, V3 and C3 regions of gp120 for determining cell tropism and coreceptor utilization. Hoffman et al., (Mol. Membr. Biol. 16:57-65, 1999), concluded the V3 loop was implicated in regulating viral tropism, but, that other regions of Env, such as the V1- and V2-loops, modulated the effects of the V3-loop. They also acknowledged that some important exceptions to this model suggested that understanding of virus tropism and Env-chemokine receptor interactions was incomplete.

Collectively, these studies point to numerous components of gp120 that may be important or essential for HIV-coreceptor interactions, and which may in fact involve multiple interactions whereby different parts of gp120 are required to work together to achieve efficient HIV-coreceptor interactions. This complexity and the proposed requirement for multiple, distant binding determinants on gp120 to form a conformational binding determinant for coreceptor usage, may be generally considered to teach away from the utility of small peptides for effectively blocking HIV-coreceptor binding interactions, particularly to block multiple HIV strains infecting different target cell types.

Moreover, while the foregoing, separate bodies of literature individually discuss models of conserved binding elements of chemokines, or of gp120, as determinants for mediating coreceptor interactions, these reports do not teach or suggest identification of conserved binding determinants shared between gp120 and chemokines. On the contrary, complex functional interactions and distinct binding mechanisms and determinants previously proposed for gp120 and chemokines may be considered to lead away from investigations aimed at identifying common binding determinants or mechanisms between gp120 and chemokines. This conclusion is well supported in the literature reviewed herein above.

Based on these and other teachings, there does not appear to be a clear suggestion in the literature to use a common blocking agent for coreceptor interactions by chemokines and HIV aimed at, or patterned after, a shared binding determinant between these two distinct molecules. The literature does not point toward any such common structural domain or binding determinant between HIV gp120 and chemokines, and the distinct structure-function reports for how HIV and chemokines are thought to interact with the subject receptors leads away from this path of inquiry.

In view of the foregoing teachings and unsolved questions in the literature, there remains an urgent need in the art for additional tools and methods to combat HIV infection and related disease. Related to this fundamental goal, there remains a clear need for additional therapeutic agents and methods targeting HIV viral entry into host cells, preferably that will include compositions and methods to block gp120 binding to different coreceptors to inhibit viral entry and infection and ameliorate HIV-related disease. Surprisingly, the instant invention fulfills these objects and satisfies additional objects and advantages which will become apparent from the following description.

### SUMMARY OF THE INVENTION

The instant invention provides novel methods and compositions for inhibiting interactions between human immunodeficiency viruses (HIVs) and chemokine receptors, termed “viral coreceptors” in this context. The methods of the invention generally comprise exposing a CXCR4 or CCR5 coreceptor of a subject to an effective amount of an anti-coreceptor binding agent of the invention to inhibit binding of the coreceptor by an HIV virus or viral protein. Typically, the anti-coreceptor binding agent is a gp120 peptide, peptide analog or mimetic that specifically binds the coreceptor.

Within certain methods of the invention, the subject is an isolated or bound coreceptor, a membrane or cell preparation comprising the coreceptor, a cell population, tissue or organ expressing the coreceptor, or a mammalian patient. In more detailed embodiments, the subject comprises a cell population, tissue or organ selected for in vivo or ex vivo treatment or diagnostic processing. Alternatively, the subject may be a mammalian patient susceptible to HIV infection and the anti-coreceptor binding agent is administered in a prophylactic or therapeutically effective dose to prevent or inhibit HIV binding to a susceptible cell and thereby preventing or inhibiting infection or a related disease condition or symptom.

In typical aspects of the invention, the anti-coreceptor binding agent is administered to the subject in an amount effective to inhibit one or more biological activities mediated by or associated with HIV-coreceptor interactions selected from (a) direct co-receptor, e.g., CXCR4 and/or CCR5, binding by HIV virus, (b) coreceptor binding by a HIV gp120 protein or a peptide fragment or derivative thereof, (c) HIV fusion with target host cells, (d) HIV virion entry into host cells, (e) HIV replication, and/or (f) HIV cell-cell or host-host transmission. In more specific embodiments, the anti-coreceptor binding agent is an HIV-1 peptide, peptide analog or mimetic and is administered to the subject in an amount effective to inhibit one or more biological activities selected from (a) direct co-receptor, i.e., CXCR4 and/or CCR5, binding by HIV-1 virus, (b) coreceptor binding by a HIV-1 gp120 protein or a peptide fragment or derivative thereof, (c) HIV-1 fusion with target host cells, (d) HIV-1 virion entry into host cells, (e) HIV-1 replication, and/or (f) HIV-1 cell-cell or host-host transmission.

Often, the anti-coreceptor binding agent is an HIV-1 peptide, peptide analog or mimetic administered to a mammalian patient in a prophylactically or therapeutically effective dose to prevent or inhibit HIV-1 binding infection, to a susceptible host, HIV-1 or an HIV-1-related disease condition or symptom.

In more detailed methods and compositions of the invention, the gp120 peptide, peptide analog or mimetic is between about 12 and about 24 amino acid residues in length and comprises a conserved CXXXXXXW amino acid sequence motif identified within the amino acid sequence of gp120 proteins of HIV isolates and also among diverse chemokines, wherein X is any naturally occurring or synthetic amino acid or amino acid analog. The peptide, peptide analog or mimetic can be modified in a wide variety of ways and forms, e.g., by addition, admixture, or conjugation of additional amino acids, peptides, proteins, chemical reagents or moieties which do not substantially alter the anti-coreceptor binding activity of the peptide.

Often, the anti-coreceptor binding agent of the invention is a peptide comprising an allelic variant that is found among native HIV gp120 peptide sequences.

Within more detailed embodiments, the anti-coreceptor binding agent is a peptide of between about 12-17 amino acids in length that includes a conserved “CXXXXXXW” amino acid sequence motif, which is selected from an exemplary “reference” peptide designated 15K comprising an amino acid sequence IRKAHCNISRAKWND (SEQ ID NO:8), or is alternatively represented by a corresponding or overlapping native peptide sequence or peptide analog that shares substantial sequence identity to the reference amino acid sequence of 15K. In various specific embodiments, the peptide includes one or more residues occurring naturally or by substitution at a relative, aligned position corresponding to a designated position for peptide 15K, selected from:

Position 1—I, M, K, S, T, L, A, V, R, P, or N;

Position 2—R, G, E, K, S, T, or I;

Position 3—Q, K, R, L, E, P, A, V, S, T, H, or D;

Position 4—A, T, P, V, E, or S;

Position 5—H, Y, F, Q. N, I, or V;

Position 7—N, D, H, T, K, E, S, I, Q, V, G, or A;

Position 8—I, L, V, Y, D, A;

Position 9—S, N, D, T, K, Y, I, or P;

Position 10—R, K, G, S, A, E, D, I, T, W, or N;

Position 11—A, R, K, T, S, G, E, D, N, Q, H, V, I, or L;

Position 12—K, D, R, E, K, Q, N, T, S, G, A, V, L;

Position 14—N, Q, D, E, K, R, A, S, T, G, M, Y, I, H, or V; and/or

Position 15—D, N, K, E, T, Q, R, S, A, I, M, or P.

In yet additional embodiments of the invention, the anti-coreceptor binding agent exhibits multi-tropic activity characterized by effective inhibition of HIV viral, or gp120 protein or peptide binding to multiple, CXCR4 and CCR5, coreceptors. Often, the multi-tropic anti-coreceptor binding agent is an HIV-1 peptide, peptide analog or mimetic administered to the subject in an amount effective to inhibit one or more biological activities of both T cell tropic (lymphotropic) and macrophage tropic (m-tropic) HIV-1 viruses selected from (a) direct co-receptor binding by viruses, (b) coreceptor binding by viral gp120 proteins or peptide fragments or derivatives thereof, (c) viral fusion with target host cells, (d) virion entry into host cells, (e) viral replication, and/or (f) viral cell-cell or host-host transmission.

Within the methods and compositions of the invention, the anti-coreceptor binding agent may be formulated in various combinations with a pharmaceutically acceptable carrier, diluent, excipient, adjuvant or other active or inactive agents, in an amount or dosage form sufficient to prevent, reduce or even alleviate HIV infection or related disease conditions or symptoms.

In yet additional aspects of the invention, the anti-coreceptor binding agent of the invention is administered according to the foregoing methods in a combinatorial formulation or coordinate treatment with one or more additional anti-HIV, antibacterial, antiviral or other therapeutically active agent(s). Within related methods and compositions, the anti-coreceptor binding agent is admixed or co-administered, simultaneously or sequentially with one or more additional anti-HIV, antibacterial, antiviral or other therapeutically active agent(s) to prevent, reduce or even alleviate HIV infection or related conditions in a mammalian patient.

The instant invention also includes kits, packages and multicontainer units containing the anti-coreceptor binding agent, optionally with other active or inactive ingredients, and/or means for administering the same for use in the diagnosis, management and/or prevention and treatment of HIV and related conditions. Typically, these kits include a diagnostic or pharmaceutical preparation of the anti-coreceptor binding agent, typically formulated with a biologically suitable carrier and optionally contained in a bulk dispensing container or unit or multi-unit dosage form. Optional dispensing means can be provided, for example an intranasal spray applicator. Packaging materials optionally include a label or instruction which indicates a desired use of the kit as described herein below.

Additional aspects of the invention include polynucleotide molecules and vector constructs encoding anti-coreceptor binding peptides and peptide analogs. Also provided are peptide vaccines and other immunogenic compositions that elicit an immune response involving production of antibodies targeting one or more epitopes of gp120 recognized by antibodies that specifically bind an anti-coreceptor binding peptide of the invention. In addition, the invention provides, antibodies, including monoclonal antibodies, and immunotherapeutic methods and compositions comprising such antibodies that specifically recognize anti-coreceptor binding peptides of the invention, for use as diagnostic and therapeutic reagents. Also provided within the invention are a variety of additional diagnostic and therapeutic tools and reagents as set forth in detail in the following description.

### BRIEF DESCRIPTION OF THE DRAWINGS

FIG. 1 demonstrates inhibition of specific chemokine binding to CXCR4 and CCRS expressing cells by exemplary HIV gp120-derived anti-coreceptor binding peptides 15K and 15D.

FIG. 2 documents inhibition of the chemotactic response of HEK-CCR5 cells to the chemokine RANTES by the exemplary anti-coreceptor binding peptides 15K and 15D. Migration of HEK-CCR5 cells to RANTES alone, or the combination of RANTES and designated concentrations of either 15K or 15D peptides, were determined in micro-chemotaxis chambers. The 15K and 15D peptides did not induce detectable chemotaxis when tested at any concentration. The results are representative of three independent experiments. *p<0.05.

FIG. 3 demonstrates inhibition of monocyte-tropic HIV-1 infection of susceptible target cells by the exemplary anti-coreceptor binding peptides 15K and l5D. Monocyte-derived macrophages were treated with the designated concentrations of the indicated peptide for a period of 1 hr prior to addition of HIV-1_{JRFL}. After 2 hr, cells were washed, and viral replication was determined after 72 hr by p24 analysis. Results are representative of 4 independent experiments.

FIG. 4 shows inhibition of T cell-tropic HIV-1 infection by the exemplary anti-coreceptor binding peptides 15K, l5D and scrambled peptide 15KS. Peripheral blood mononuclear cells were treated with designated concentrations of peptide for a period of 1 hr prior to addition of HIV-1_{IIIB}. After 2 hr, cells were washed, and viral replication was determined after 48 hr by analysis of p24 levels. Results are representative of 4 independent experiments.

FIGS. 5A through 5C demonstrate the effect of peptides 15K, 15D and scrambled peptide 15KS on the binding of anti-CXCR4 antibody 12G5 to CEMx174 cells. FIG. 5A. Dose dependent effect of peptide 15K in comparison with SDF-1α. FIG. 5B. The comparison of the effects of 15K and 15D. FIG. 5C. The comparison of the effects of 15K and 15KS. Cells were preincubated at 22° C. with peptides at designated concentrations of SDF-1α for 60 min. Then cells were incubated with FITC-labeled anti-human CXCR4 monoclonal antibodies 12G5 for 40 min at 22° C. and washed with FACS buffer prior to flow cytometry.

FIGS. 6A through 6D depict the effect of peptide pre-treatment on the mobilization of intracellular Ca²⁺ in THP-1 cells in response to SDF-1α. Cells loaded with fura-2 were preincubated with peptide 15K (FIG. 6C), 15D (FIG. 6B) and 15CW (FIG. 6D) at a final concentration of 500 μM for 3 min prior to stimulation with SDF-1α (100 ng/ml).

FIGS. 7A through 7C depicts the inhibition of chemokine receptor ligand binding by peptide pre-treatment. FIG. 7A. Effect of pre-treatment with peptides 15K and 15D on the binding of MIP-1β to SupT1/CCR5 cells; FIG. 7B. Effect of pre-treatment with peptides 15K and 1SD on the binding of SDF-1α to CEMx174 cells; and FIG. 7C. Effect of pre-treatment with 15K and control peptide 15GIG on the binding of SDF-1α to CEmx174 cells. Binding studies were performed as described in Materials and Methods.

### DETAILED DESCRIPTION

The present invention provides novel methods and compositions for clinical and diagnostic use in the evaluation, treatment and prevention of HIV infection and related disease conditions by reducing the ability of HIV to bind and fuse to cells. Typically, the compositions and procedures of the invention are directed toward prophylaxis or treatment of HIV-1 infection and related clinical conditions. The methods of the invention involve administering an effective amount of an anti-coreceptor binding agent to a subject to inhibit HIV-coreceptor interaction. Typically, the anti-coreceptor binding agent is administered in a prophylactic or therapeutically effective dose to a mammalian patient susceptible to HIV infection. Alternatively, the subject may comprise a susceptible cell population, tissue or organ, selected for in vivo or ex vivo treatment or diagnostic processing involving exposure of the subject to an anti-coreceptor binding agent (for example, bone marrow or other tissue or organ materials treated ex vivo before re-implantation or transplant).

The methods and compositions of the invention involve delivery or formulation of an anti-coreceptor binding agent in an amount that is effective to inhibit HIV-coreceptor interactions, and/or to inhibit one or more selected biological activities or conditions associated with, or mediated by, HIV-coreceptor interactions. Selected biological activities include direct co-receptor binding by HIV viruses, coreceptor binding by selected viral proteins or peptides (including gp120 proteins and peptide fragments or derivatives of gp120), as well as binding by antibodies that recognize epitopes on gp120 or on a selected chemokine or HIV coreceptor. Additional biological activities in this context include HIV infection and related activities and conditions, for example HIV fusion with target host cells, HIV virion entry into host cells, HIV propagation and related HIV infective events, as well as specific HIV-related disease conditions including the full range of clinical conditions and disease states associated with AIDS and AIDS Related Complex (ARC) (for example, Kaposi's sarcoma and opportunistic viral (e.g., herpes) and bacterial (e.g., pneumonia infections).

As used herein, the term “anti-coreceptor binding agent” is meant to include anti-coreceptor binding HIV peptides, as well as peptide analogs and peptide mimetics which exhibit comparable, or substantially the same, anti-coreceptor binding activity as a selected anti-coreceptor binding HIV peptide as described herein. The term anti-coreceptor binding peptide includes all of the reference peptides described herein, as well as other natural or artificially selected mutant or allelic forms and derivatives of these reference peptides having the desired anti-coreceptor binding activity. The term peptide analog refers to such artificially modified peptide analogs as chemically cleaved peptide fragments, chemically modified peptide derivatives, site directed mutant peptide variants having one or more amino acid insertions, substitutions or deletions, and the like.

The anti-coreceptor binding peptides, analogs and mimetics of the invention function to specifically inhibit HIV-1 gp120 binding interactions with chemokine receptors (HIV-1 coreceptors). In addition, the anti-coreceptor binding agents are effective to inhibit or block HIV-1 cell fusion and virion entry, thereby impairing viral replication and transmission. Correlated with these activities, the anti-coreceptor binding agents and methods of the invention provide for safe and effective treatment of HIV-1 infection and related diseases. Ancillary uses are also readily implemented using these compositions and methods for diagnosing and evaluating HIV infection and related activities and disease mechanisms.

The description herein illustrates production and characterization of peptides modeled after a novel structural motif identified in the HIV-1 gp120 envelope protein. This novel structural motif shares similarities with a corresponding structural motif identified as a conservative feature among diverse chemokines. This conservative motif is represented in one aspect by the reference sequences identified for HIV strains HXB2, IIIB, and JRFL, which represent operable anti-coreceptor binding peptides within the invention provided as native fragments of the corresponding gp120 proteins of these strains. As shown in Table 1, below, these sequences aligned with MIP-1β and SDF-1α embrace a novel, HIV-1 coreceptor binding motif.

TABLE

AMINO ACID ALIGNMENT OF PARTIAL SEQUENCES OF GP120
FROM REPRESENTATIVE HIV STRAINS WITH PARTIAL
SEQUENCES OF MIP-1β AND SDF-1α

[symbol]

| | | |
|---|---|---|
| HIV | | |
| strain* | 326 340 | |
| HXB2 | M R Q A H C N I S R A K W N N | SEQ ID NO:1 |
| | 326 340 | |
| III B | M R Q A H C N I S R A K W N A | SEQ ID NO:2 |
| | 322 336 | |
| JR FL | I R Q A H C N I S R A K W N D | SEQ ID NO:3 |
| Consensus | I R Q A H C N V S R S E W N K | SEQ ID NO:4 |
| HIV A | | |
| Consensus | I R Q A H C N I S R A Q W N N | SEQ ID NO:5 |
| HIV B | | |
| chemokine | 47 60 | |
| MIP-1β | S K Q - V C A D P S E S W V Q | SEQ ID NO:6 |
| | 48 61 | |
| SDF-1α | N R Q - V C I D P K L K W I Q | SEQ ID NO:7 |

*The residues numbered according to (Korber, B. and Los Alamos National Laboratory - Theoretical Biology and Biophysics Group T-10, Human retroviruses and AIDS. 1998: a compilation and analysis of nucleic acid and amino acid sequences. Published by Theoretical Biology and Biophysics Group T-10 Los Alamos National Laboratory, Los Alamos, N.M., 1998, incorporated herein by reference). The residues that are shared between identified regions of HIV-1 gp120 and chemokines are shown in bold.

This alignment illustrates the finding herein that the amino acid sequences of nearly all chemokines feature a Trp residue located at the beginning of a C-terminal α-helix and separated by six intervening residues from a conserved Cys residue. Comparing this conserved motif with the gp120 molecule of HIV-1, it is further demonstrated that all HIV isolates have a similar structural motif adjacent the V3 loop. As described in further detail below, computer modeling based on the sequence of HIV-1 strain JRFL which closely relates to a Consensus HIV-1 sequence, demonstrates that a fragment of gp120 containing a conserved “CXXXXXXW motif” can be “template forced” onto the homologous portion of the chemokine MIP-1β.

Thus, the anti-coreceptor binding peptides of the invention typically feature the above noted CXXXXXXW motif, with additional amino acids, peptides, proteins, chemical reagents or moieties combined or conjugated therewith. These peptides include native HIV-1 peptides, such as those identified above in Table 1 and other known HIV-1 peptides that correspond to, or include, a partial or complete, homologous sequence to these exemplary peptides. Other peptides comprising the novel structural motif that includes the above noted CXXXXXXW anti-coreceptor binding determinant can comprise allelic variants among native peptide sequences, or synthetic or mutant peptide analogs of selected native HIV-1 gp120 peptides.

Two exemplary peptides within the invention, designated 15K and 15D, include the C-terminal part of the V3 loop of gp120 and the N-terminal part of the C3 segment of the protein (Table 2). These peptides differ from each other by the presence of a D or K (Asp or Lys) residue at the twelfth position, which difference reflects allelic sequence variation among natural HIV-1 isolates. In addition, both of these exemplary synthetic peptides differ from corresponding, native HIV-1 sequences by a site-directed mutation comprising a substitution of K for Q (Lys for Gln) at the third residue position in the peptides, which reflects allelic sequence variation among natural HIV-1 isolates and also results in reduced susceptibility of the peptides to hydrolysis.

TABLE 2
SEQUENCES OF EXEMPLARY SYNTHETIC PEPTIDES

| | |
|---|---|
| Design- | |
| tion | Sequence |

| | | |
|---|---|---|
| 15K | I R K A H C N I S R A K W N D | (SEQ ID NO:8) |
| 15D | I R K A H C N I S R A D W N D | (SEQ ID NO:9) |

These exemplary synthetic peptides of the invention inhibit or block binding or “docking” interactions between the HIV-1 envelope protein gp120 and chemokine receptors (e.g., CXCR4 and CCR5) that function as “coreceptors” for HIV entry on the surface of target cells (macrophages and T lymphocytes). The natural ligands of the coreceptors are chemokines (e.g., SDF-1α and MIP-1β), and it is further demonstrated herein that the conserved structural elements identified for the HIV gp120 protein motif that are shared with chemokines mediate critical HIV-coreceptor interactions. Detailed functional analyses of the peptides show that both are effective in competing with chemokines for binding to CCR5- and CXCR4-expressing cells. The peptides also inhibit monocyte chemotaxis stimulated by the chemokine RANTES (Regulated upon Activation Normal T-cell Expressed and Secreted).

In addition, the anti-coreceptor binding peptides of the invention are shown herein to be potent inhibitors of HIV replication. In certain aspects, the peptides effect this inhibition in a multi-tropic or multi-specific manner to prevent or treat infection by both T cell tropic (lymphotropic) and macrophage tropic (m-tropic) HIV strains by blocking HIV interactions with distinct (CXCR4 and CCR5) coreceptors. This ability to use small peptides to achieve multi-tropic (i.e., multi-receptor) blockade is a surprising and important advantage satisfied by the invention.

The anti-coreceptor binding peptides of the present invention include naturally occurring peptide variants, e.g., naturally occurring allelic variants and mutant proteins, as well as synthetic, e.g., chemically or recombinantly engineered, peptide fragments and analogs. As used herein, anti-coreceptor binding peptide “analogs” is meant to include a modified gp120 peptide incorporating one or more amino acid substitutions, insertions, rearrangements or deletions as compared to a native amino acid sequence of an HIV-1 gp120 anti-coreceptor binding peptide domain, fragment or motif, as described herein. Anti-coreceptor binding peptide analogs thus modified exhibit substantial anti-coreceptor binding activity comparable to that of a corresponding native peptide, which is activity that at least 50%, typically at least 75% or greater, compared to activity of the corresponding native peptide (e.g., as determined by an in vitro coreceptor binding assay or HIV-1 infection assay). As used herein, the term “biologically active anti-coreceptor binding analogs and mimetics” refers to analogs or mimetics of native peptides which encompass the entire length, sequence or chemical structure of the corresponding native peptide but which nevertheless maintains substantial anti-coreceptor binding activity as described above in an appropriate assay system.

For purposes of the present invention, the term anti-coreceptor binding peptide “analog” thus includes derivatives or synthetic variants of a native HIV-1 gp120 anti-coreceptor binding peptide, such as amino and/or carboxyl terminal deletions and fusions, as well as intrasequence insertions, substitutions or deletions of single or multiple amino acids. Insertional amino acid sequence variants are those in which one or more amino acid residues are introduced into a predetermined site in the protein. Random insertion is also possible with suitable screening of the resulting product. Deletional variants are characterized by removal of one or more amino acids from the sequence. Substitutional amino acid variants are those in which at least one residue in the sequence has been removed and a different residue inserted in its place.

Where a native HIV-1 gp120 anti-coreceptor binding peptide is modified by amino acid substitution, amino acids are generally replaced by other amino acids having similar, conservatively related chemical properties such as hydrophobicity, hydrophilicity, electronegativity, bulky side chains and the like. Residue positions which are not identical to the native peptide sequence are thus replaced by amino acids having similar chemical properties, such as charge or polarity, which changes are not likely to substantially effect the properties of the peptide analog. These and other minor alterations substantially maintain the immunoidentity (e.g., recognition by one or more monoclonal antibodies that recognize a native HIV-1 gp120 anti-coreceptor binding peptide) and other biological activities of the native peptide.

In this context, the term “conservative amino acid substitution” refers to the general interchangeability of amino acid residues having similar side chains. For example, a group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

Anti-coreceptor binding peptide analogs also include modified forms of a native HIV-1 gp120 anti-coreceptor binding peptide incorporating stereoisomers (e.g., D-amino acids) of the twenty conventional amino acids, or unnatural amino acids such as a, α-disubstituted amino acids, N-alkyl amino acids, lactic acid. These and other unconventional amino acids may also be substituted or inserted within native HIV-1 gp120 anti-coreceptor binding peptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and amino acids (e.g., 4-hydroxyproline). For purposes of the present invention, analogs of native HIV-1 gp120 anti-coreceptor binding peptides also include single or multiple substitutions, deletions and/or additions of carbohydrate, lipid and/or proteinaceous moieties that occur naturally or artificially as structural components of gp120 peptides or are bound or otherwise associated with the peptide analog.

To facilitate production and use of anti-coreceptor binding peptide analogs within the invention, reference can be made to molecular phylogenetic studies that characterize conserved and divergent protein structural and functional elements between different HIV-1 strains, and between HIV-1 and other HIV taxa and more distantly related retroviruses. In this regard, available studies provide detailed assessments of gp120 protein structure-function relationships on a fine molecular level. These studies include detailed sequence comparisons identifying conserved and divergent structural elements among a large number of HIV-1 gp120 allelic variants, for example. Each of these conserved and divergent structural elements facilitate practice of the invention by pointing to useful targets that for modifying native HIV-1 gp120 anti-coreceptor binding peptides to confer desired structural and/or functional changes.

In this context, existing sequence alignments may be analyzed, or conventional sequence alignment methods may be employed to yield sequence comparisons for analysis, to identify corresponding protein regions and amino acid positions between native HIV-1 gp120 anti-coreceptor binding peptides and related or homologous peptides bearing a structural element of interest for incorporation within an anti-coreceptor binding peptide analog. Typically, one or more amino acid residues marking a structural element of interest in a different reference peptide sequence is incorporated within the anti-coreceptor binding peptide analog. For example, a cDNA encoding a native HIV-1 gp120 anti-coreceptor binding peptide may be recombinantly modified at one or more corresponding amino acid position(s) (i.e., corresponding positions that match or span a similar aligned sequence element according to accepted alignment methods to residues marking the structural element of interest in a heterologous reference peptide sequence) to encode an amino acid deletion, substitution, or insertion that alters corresponding residue(s) in the native HIV-1 gp120 anti-coreceptor binding peptide to generate an operable peptide analog within the invention having an analogous structural and/or functional element as the reference peptide or protein.

Within this rational design method for constructing anti-coreceptor binding peptide analogs, the native or wild-type identity of residue(s) at amino acid positions corresponding to a structural element of interest in a heterologous reference peptide or protein may be altered to the same, or a conservatively related, residue identity as the corresponding amino acid residue(s) in the reference peptide or protein. However, it is often possible to alter native amino acid residues non-conservatively with respect to the corresponding reference protein residue(s). In particular, many non-conservative amino acid substitutions, particularly at divergent sites suggested to be more amenable to modification, may yield a moderate impairment or neutral effect, or even enhance a selected biological activity, compared to the function of a native HIV-1 gp120 anti-coreceptor binding peptide.

Extensive protein structure-function data are available in the literature pertaining to HIV that will serve and guide the artisan to prepare functional anti-coreceptor binding peptide analogs for use within the invention. Structure-function relationships between HIV-1 strains and between HIV-1 and more distantly related retroviruses may be elucidated using conventional molecular phylogenetic analysis coupled with functional assays for determining anti-coreceptor binding activities described herein. Sequence alignment and comparisons to forecast useful peptide analogs and mimetics will be further refined by analysis of crystalline structure (see, e.g., Löebermann et al., J. Mol. Biol. 177:531-556, 1984; Huber et al., Biochemistry 28:8951-8966, 1989; Stein et al., Nature 347:99-102, 1990; Wei et al., Structural Biology 1:251-255, 1994, each incorporated herein by reference) coupled with computer modeling methods known in the art. These analyses allow detailed structure-function mapping to identify desired structural elements and modifications for incorporation into anti-coreceptor binding peptide analogs and mimetics that will exhibit substantial anti-coreceptor binding activity for use within the methods of the invention.

Native HIV-1 gp120 anti-coreceptor binding peptides and peptide analogs within the invention are typically between about 6-35 amino acid residues in length, more typically between about 10 and 21 or 22 amino acid residues in length. Within certain embodiments, the native peptides and analogs are between about 10-17 residues in length. In more specific embodiments, peptides are 10, 12, 13, 15, 17, 21, or 22 amino acid residues in length.

Anti-coreceptor binding peptide analogs of the invention typically show substantial sequence identity to a corresponding native HIV-1 gp120 anti-coreceptor binding peptide sequence. Within the foregoing length ranges, both the native HIV-1 gp120 anti-coreceptor binding peptide and the peptide analogs typically comprise the conserved “CXXXXXXW” motif, described above, which may be extended to include additional residues from the native HIV-1 gp120 anti-coreceptor binding peptide sequence or non-native residues, fusion protein members, chemical moieties and the like. As applied to anti-coreceptor binding peptide analogs and fragments, these analogs and fragments typically exhibit substantial amino acid sequence identity to a corresponding native HIV-1 gp120 anti-coreceptor binding peptide sequence.

The term “substantial sequence identity” as used herein means that the two subject amino acid sequences, when optimally aligned, such as by the programs GAP or BESTFIT using default gap penalties, share at least 80 percent sequence identity, often at least 90-95 percent or greater sequence identity. “Percentage amino acid identity” refers to a comparison of the amino acid sequences of two peptides which, when optimally aligned, have approximately the designated percentage of the same amino acids. Sequence comparisons are generally made to a reference sequence over a comparison window of at least 10 residue positions, frequently over a window of at least 15-20 amino acids, wherein the percentage of sequence identity is calculated by comparing a reference sequence to a second sequence, the latter of which may represent, for example, a peptide analog sequence that includes one or more deletions, substitutions or additions which total 20 percent, typically less than 5-10% of the reference sequence over the window of comparison. The reference sequence may be a subset of a larger sequence, for example, as a segment of the HIV-1 gp120 protein. Optimal alignment of sequences for aligning a comparison window may be conducted according to the local homology algorithm of Smith and Waterman (Adv. Appl. Math. 2:482, 1981), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), or by computerized implementations of these algorithms (GAP, BESTFIT, FASTA, and TFASTA in the Wisconsin Genetics Software Package Release 7.0, Genetics Computer Group, 575 Science Dr., Madison, Wis.).

By aligning a peptide analog optimally with a corresponding native HIV-1 gp120 anti-coreceptor binding peptide, and by using appropriate assays, e.g., coreceptor binding assays, to determine a selected biological activity, one can readily identify operable peptide analogs for use within the methods and compositions of the invention. Anti-coreceptor binding peptide analogs are typically specifically immunoreactive with antibodies raised to the corresponding native HIV-1 gp120 anti-coreceptor binding peptide. Likewise, nucleic acids encoding functional anti-coreceptor binding peptide analogs will typically selectively hybridize to nucleic acid sequences encoding a corresponding native HIV-1 gp120 anti-coreceptor binding peptide under accepted, moderate or high stringency hybridization conditions (see, e.g., Sambrook et al., Molecular Cloning: A Laboratory Manual, 2nd ed., Cold Spring Harbor Laboratories, Cold Spring Harbor, N.Y., 1989, incorporated herein by reference).

The phrase “selectively hybridizing to” refers to a selective interaction between a nucleic acid probe that hybridizes, duplexes or binds preferentially to a particular target DNA or RNA sequence, for example when the target sequence is present in a heterogenous preparation such as total cellular DNA or RNA. Generally, nucleic acid sequences encoding functional anti-coreceptor binding peptide analogs and fragments will hybridize to nucleic acid sequences encoding native HIV-1 gp120 anti-coreceptor binding peptides under stringent conditions selected to be about 5° C. lower than the thermal melting point (Tₘ) for the subject sequence at a defined ionic strength and pH. The Tₘ is the temperature (under defined ionic strength and pH) at which 50% of the complementary or target sequence hybridizes to a perfectly matched probe. For discussions of nucleic acid probe design and annealing conditions, see, for example, Sambrook et al., Molecular Cloning: A Laboratory Manual, 2nd ed., Cold Spring Harbor Laboratory, 1989 or Current Protocols in Molecular Biology F. Ausubel et al, ed., Greene Publishing and Wiley-Interscience, New York, 1987, each of which is incorporated herein by reference. Typically, stringent or selective conditions will be those in which the salt concentration is at least about 0.02 molar at pH 7 and the temperature is at least about 60° C. Less stringent selective hybridization conditions can also be chosen. As other factors may significantly affect the stringency of hybridization, including, among others, base composition and size of the complementary strands, the presence of organic solvents and the extent of base mismatching, the combination of parameters is more important than the specific measure of any one.

Anti-coreceptor binding peptide analogs of the invention typically show substantial sequence identity to a corresponding native HIV1 gp120 anti-coreceptor binding peptide sequence. Within the foregoing length ranges, both the native HIV-1 gp120 anti-coreceptor binding peptide and the peptide analogs typically comprise the conserved “CXXXXXXW” motif, described above, which may be extended to include additional residues from the native HIV-1 gp120 anti-coreceptor binding peptide sequence, non-native residues, fusion protein members, chemical moieties, and the like. As applied to anti-coreceptor binding peptide analogs and fragments, these analogs and fragments typically exhibit substantial amino acid sequence identity to a corresponding native HIV-1 gp120 anti-coreceptor binding peptide sequence.

Various peptide analogs are contemplated within the scope of the invention, which typically satisfy the foregoing length criteria of 10 to and include the conserved “CXXXXXXW” motif, but which incorporate selected sequence modifications at one or more positions to yield a desired structural or activity modification. To illustrate the breadth of different peptides and peptide analogs that are useful within the invention, the following table (Table 3) provides a map of anti-coreceptor binding peptide sequence variants based on a detailed analysis of different strains of HIV-1, along with structure-function analysis directed to both gp120 and chemokine structure-function, further guided by general rules of peptide structure-function.

TABLE 3

MENU FOR CONSTRUCTING OPERABLE HIV gp120
PEPTIDES AND PEPTIDE ANALOGS

1 2 3 4 5 6 7 8 9 10 11 12 13 14 15

| | | |
|---|---|---|
| Peptide 15K | I R Q A H C N I S R A K W N D | SEQ ID NO:8 |
| Peptide 15D | D | SEQ ID NO:9 |
| | M G K T Y D L N K R R Q N | |
| | K E R P F H V D G K E D K | |
| | S K L V Q T Y T S T D E E | |
| | T S E S N K D K A S Q K T | |
| | L T P I E A Y E G N R Q | |
| | A I A V S I D E T A R | |
| | V V I P I D S S S | |
| | A S Q T N G T A | |
| | P T V W Q A G I | |
| | N H G N H V M M | |
| | D A V L Y P | |
| | I I | |
| | H H | |
| | V | |

In accordance with the foregoing table, peptides which satisfy the above-described length criteria and include the conserved “CXXXXW” motif can also be selected from corresponding or overlapping native peptides among any known HIV isolate having a variant sequence as compared to a corresponding “reference” peptide. As illustrated in Table 3, one reference peptide that may be used in this context is the 15K peptide, which provides an exemplary reference sequence for comparison with other peptide sequences within the invention. Against this reference sequence, the table provides a range of amino acid residues that can be effectively substituted at the indicated residue positions (numbered in reference to the 15K peptide) which, for the substitutions designated by regular font single amino acid code, follow natural variations in the HIV gp120 available in the various HIV sequence data banks (e.g., http://hiv-web.lanl.gov/., incorporated herein by reference). Each of these residues can be incorporated, in a native HIV gp120-derived peptide, or in a single- or multiple-substituted peptide analog of the invention, to yield effective anti-coreceptor binding agents.

Thus, peptides and peptide analogs are provided which satisfy the above-described length criteria and include the conserved “CXXXXXXW” motif, and which include any one, or any combination, of the following alternative residues occurring naturally or by substitution at the indicated positions (as designated for peptide 15K in Table 3 and determined for the subject peptide by conventional comparison and alignment against, e.g., the 15K reference sequence, as described herein): Position 1—I, M, K, S, T, L, A, V, R, P, or N; Position 2—R, G, E, K, S, T, or I; Position 3—Q, K, R, L, E, P, A, V, S, T, H, or D; Position 4—A, T, P, V, E, or S; Position 5—H, Y, F, Q, N, I, or V; Position 7—N, D, H, T, K, E, S, I, Q, V, G, or A; Position 8—I, L, V, Y, D, A; Position 9—S, N+EE, D, T, K, Y, I, or P; Position 10—R, K, G, S, A, E, D, I, T, W, or N; Position 11—A, R, K, T, S, G, E, D, N, Q, H, V, I, or L; Position 12—K, D, R, E, K, Q, N, T, S, G, A, V, L; Position 14—N, Q, D, E, K, R, A, S, T, G, M, Y, I, H, or V; Position 15—D, N, K, E, T, Q, R, S, A, I, M, or P.

Certain of the foregoing residue/position alternatives, for example, the residue/position alternatives designated by underscoring in the preceding paragraph, may be selected in a greater number of peptides and analogs of the invention compared to other alternatives. This preference may be guided based on the greater degree of conservation of the indicated residue at corresponding positions among different HIV-1 isolates, or based on a convergent or homologous occurrence of the indicated residue at corresponding positions in gp120 of a selected HIV isolate and in a selected chemokine. Additional selections within the foregoing “menu” will be guided to favor conservative structural relationships between original and substitute residues in the preparation of anti-coreceptor binding peptide analogs, as described herein. In accordance with these selection principals, the above-noted, alternative residues may be included in peptides and peptide analogs of the invention, singly or in any combination of 2, 3, 4 or more and up to 13 residues (e.g., as exemplified by the various combinations shown in the separate lines of Table 3), selected or substituted at the indicated positions.

Within additional aspects of the invention, peptide mimetics are provided which comprise a peptide or non-peptide molecule that mimics the tertiary binding structure and activity of the anti-coreceptor binding peptides described herein. These peptide mimetics include recombinantly or chemically modified peptides, as well as non-peptide anti-coreceptor binding agents such as small molecule drug mimetics, as further described below.

In one aspect, peptides of the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di (lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

The peptide compounds of the invention, including peptidomimetics, can also be covalently bound to one or more of a variety of nonproteinaceous polymers, e.g., polyethylene glycol, polypropylene glycol, or polyoxyalkenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337, all which-are incorporated by reference in their entirety herein.

Other peptide analogs and mimetics within the invention include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins, e.g., immunogenic moieties may also be employed.

In certain embodiments, glycosylation alterations of anti-coreceptor binding agents are included, which can be made, e.g., by modifying the glycosylation patterns of a peptide during its synthesis and processing, or in further processing steps. Particularly preferred means for accomplishing this are by exposing the peptide to glycosylating enzymes derived from cells which normally provide such processing, e.g., mammalian glycosylation enzymes. Deglycosylation enzymes are also contemplated. Also embraced are versions of the same primary amino acid sequence which have other minor modifications, including phosphorylated amino acid residues, e.g., phosphotyrosine, phosphoserine, or phosphothreonine, or other moieties, including ribosyl groups or cross-linking reagents.

Peptidomimetics may also have amino acid residues which have been chemically modified by phosphorylation, sulfonation, biotinylation, or the addition or removal of other moieties, particularly those which have molecular shapes similar to phosphate groups. In some embodiments, the modifications will be useful labeling reagents, or serve as purification targets, e.g., affinity ligands.

A major group of peptidomimetics within the invention are covalent conjugates of the anti-coreceptor binding peptides, or fragments thereof, with other proteins or peptides. These derivatives can be synthesized in recombinant culture such as N- or C-terminal fusions or by the use of agents known in the art for their usefulness in cross-linking proteins through reactive side groups. Preferred peptide and protein derivatization sites for targeting by cross-linking agents are at free amino groups, carbohydrate moieties, and cysteine residues.

Fusion polypeptides between anti-coreceptor binding peptides and other homologous or heterologous peptides and proteins are also provided. Many growth factors and cytokines are homodimeric entities, and a repeat construct of anti-coreceptor binding peptide linked to form “cluster peptides” will yield various advantages, including lessened susceptibility to proteolytic degradation. Various alternative multimeric constructs comprising peptides of the invention are also provided. In one embodiment, various polypeptide fusions are provided as described in U.S. Pat. Nos. 6,018,026 and 5,843,725, by linking one or more anti-coreceptor binding peptides of the invention with a heterologous, multimerizing polypeptide or protein, for example, immunoglobulin heavy chain constant region, or an immunoglobulin light chain constant region. The biologically active, multimerized polypeptide fusion thus constructed can be a hetero- or homo-multimer, e.g., a heterodimer or homodimer, which may each comprise one or more distinct anti-coreceptor binding peptide(s) of the invention. Other heterologous polypeptides may be combined with the anti-coreceptor binding agents to yield fusions comprising, e.g., a hybrid protein exhibiting heterologous (e.g., CD4) receptor binding specificity. Likewise, heterologous fusions may be constructed exhibit a combination of properties or activities of the derivative proteins. Other typical examples are fusions of a reporter polypeptide, e.g., CAT or luciferase, with a peptide of the invention, to facilitate localization of the fused protein (see, e.g., Dull et al., U.S. Pat. No. 4,859,609, incorporated herein by reference). Other gene/protein fusion partners useful in this context include bacterial beta-galactosidase, trpE, Protein A, beta-lactamase, alpha amylase, alcohol dehydrogenase, and yeast alpha mating factor (see, e.g., Godowski et al., Science 241:812-816, 1988, incorporated herein by reference).

The present invention also contemplates the use of anti-coreceptor binding agents modified by covalent or aggregative association with chemical moieties. These derivatives generally fall into the three classes: (1) salts, (2) side chain and terminal residue covalent modifications, and (3) adsorption complexes, for example with cell membranes. Such covalent or aggregative derivatives are useful for various purposes, for example as immunogens, as reagents in immunoassays, or in purification methods such as for affinity purification of ligands or other binding ligands. For example, an anti-coreceptor binding agent can be immobilized by covalent bonding to a solid support such as cyanogen bromide-activated Sepharose, by methods which are well known in the art, or adsorbed onto polyolefin surfaces, with or without glutaraldehyde cross-linking, for use in the assay or purification of antibodies that specifically bind the anti-coreceptor binding agent. The anti-coreceptor binding agent can also be labeled with a detectable group, for example radioiodinated by the chloramine T procedure, covalently bound to rare earth chelates, or conjugated to another fluorescent moiety for use in diagnostic assays.

Those of skill in the art recognize that a variety of techniques are available for constructing peptide mimetics with the same or similar desired biological activity as the corresponding peptide compound but with more favorable activity than the peptide with respect to solubility, stability, and susceptibility to hydrolysis and proteolysis (see, e.g., Morgan and Gainor, Ann. Rep. Med. Chem. 24:243-252, 1989, incorporated herein by reference). The following describes methods for preparing peptide mimetics modified at the N-terminal amino group, the C-terminal carboxyl group, and/or changing ore or more of the amido linkages in the peptide to a non-amido linkage. It being understood that two or more such modifications can be coupled in one peptide mimetic structure (e.g., modification at the C-terminal carboxyl group and inclusion of a —CH₂-carbamate linkage between two amino acids in the peptide.

For N-terminal modifications, the peptides typically are synthesized as the free acid but, as noted above, can be readily prepared as the amide or ester. One can also modify the amino and/or carboxy terminus of the peptide compounds of the invention to produce other compounds of the invention. Amino terminus modifications include methylating (i.e., —NHCH₃ or —NH(CH₃)₂), acetylating, adding a carbobenzoyl group, or blocking the amino terminus with any blocking group containing a carboxylate functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints. Amino terminus modifications are as recited above and include alkylating, acetylating, adding a carbobenzoyl group, forming a succinimide group, and the like. Specifically, the N-terminal amino group can then be reacted as follows:

(a) to form an amide group of the formula RC(O)NH— where R is as defined above by reaction with an acid halide (e.g., RC(O)Cl) or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—;

(b) to form a succinimide group by reaction with succinic anhydride. As before, an approximately equimolar amount or an excess of succinic anhydride (e.g., about 5 equivalents) can be employed and the amino group is converted to the succinimide by methods well known in the art including the use of an excess (e.g., ten equivalents) of a tertiary amine such as diisopropylethylamine in a suitable inert solvent (e.g., dichloromethane) (see, for example, U.S. Pat. No. 4,612,132, incorporated herein by reference). It is understood that the succinic group can be substituted with, for example, C₂-C₆ alkyl or —SR substituents which are prepared in a conventional manner to provide for substituted succinimide at the N-terminus of the peptide. Such alkyl substituents are prepared by reaction of a lower olefin (C₂-C₆) with maleic anhydride in the manner described by (U.S. Pat. No. 4,612,132) and —SR substituents are prepared by reaction of RSH with maleic anhydride where R is as defined above;

(c) to form a benzyloxycarbonyl-NH— or a substituted benzyloxycarbonyl-NH-group by reaction with approximately an equivalent amount or an excess of CBZ-Cl (i.e., benzyloxycarbonyl chloride) or a substituted CBZ-Cl in a suitable inert diluent (e.g., dichloromethane) preferably containing a tertiary amine to scavenge the acid generated during the reaction;

(d) to form a sulfonamide group by reaction with an equivalent amount or an excess (e.g., 5 equivalents) of R—S(O)₂Cl in a suitable inert diluent (dichloromethane) to convert the terminal amine into a sulfonamide where R is as defined above. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes);

(e) to form a carbamate group by reaction with an equivalent amount or an excess (e.g., 5 equivalents) of R—OC(O)Cl or R—OC(O)OC₆H₄-p-NO₂ in a suitable inert diluent (e.g., dichloromethane) to convert the terminal amine into a carbamate where R is as defined above. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes); and

(f) to form a urea group by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. Preferably, the inert diluent contains an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group is replaced by an ester (i.e., —C(O)OR where R is as defined above), resins as used to prepare peptide acids are employed, and the side chain protected peptide is cleaved with base and the appropriate alcohol, e.g., methanol. Side chain protecting groups are then removed in the usual fashion by treatment with hydrogen fluoride to obtain the desired ester.

In preparing peptide mimetics wherein the C-terminal carboxyl group is replaced by the amide —C(O)NR₃R₄, where R₃ and R₄ independently are hydrogen or a lower alkyl, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide and reaction with an alkylamine or a dialkylamine yields a side chain protected alkylamide or dialkylamide (i.e., the C-terminus is —C(O)NRR₁ where R and R₁ independently are hydrogen or a lower alkyl). Side chain protection is then removed in the usual fashion by treatment with hydrogen fluoride to give the free amides, alkylamides, or dialkylamides.

In another alternative embodiment, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

One can also cyclize the anti-coreceptor binding peptides of the invention, or incorporate a desamino or descarboxy residue at the termini of the peptide, so that there is no terminal amino or carboxyl group, to decrease susceptibility to proteases or to restrict the conformation of the peptide. C-terminal functional groups of the compounds of the present invention include amide, amide lower alkyl, amide di (lower alkyl), lower alkoxy, hydroxy, and carboxy, and the lower ester derivatives thereof, and the pharmaceutically acceptable salts thereof.

Other methods for making peptide derivatives and mimetics of the are described in Hruby et al., (Biochem J. 268:249-262, 1990, incorporated herein by reference). According to these methods, the anti-coreceptor binding peptide compounds of the invention also serve as structural models for non-peptide mimetic compounds with similar biological activity. Those of skill in the art recognize that a variety of techniques are available for constructing compounds with the same or similar desired biological activity as the lead peptide compound but with more favorable activity than the lead with respect to solubility, stability, and susceptibility to hydrolysis and proteolysis (see, e.g., Morgan and Gainor, Ann. Rep. Med. Chem. 24:243-252, 1989, incorporated herein by reference). These techniques include replacing the peptide backbone with a backbone composed of phosphonates, amidates, carbamates, sulfonamides, secondary amines, and/or N-methylamino acids.

Peptide mimetics wherein one or more of the peptidyl linkages —C(O)NH— have been replaced by such linkages as a —CH₂-carbamate linkage, a phosphonate linkage, a —CH₂-sulfonamide linkage, a urea linkage, a secondary amine (—CH₂NH—) linkage, and an alkylated peptidyl linkage —C(O)NR₆— where R₆ is lower alkyl are prepared during conventional peptide synthesis by merely substituting a suitably protected amino acid analogue for the amino acid reagent at the appropriate point during synthesis. Suitable reagents include, for example, amino acid analogues wherein the carboxyl group of the amino acid has been replaced with a moiety suitable for forming one of the above linkages. For example, if one desires to replace a —C(O)NR— linkage in the peptide with a —CH₂-carbamate linkage (—CH₂OC(O)NR—), then the carboxyl (—COOH) group of a suitably protected amino acid is first reduced to the —CH₂OH group which is then converted by conventional methods to a —OC(O)Cl functionality or a para-nitrocarbonate —OC(O)O—C₆H₄-p-NO₂ functionality. Reaction of either of such functional groups with the free amine or an alkylated amine on the N-terminus of the partially fabricated peptide found on the solid support leads to the formation of a —CH₂OC(O)NR— linkage. For a more detailed description of the formation of such —CH₂-carbamate linkages, see, e.g., Cho et al., (Science 261:1303-1305, 1993, incorporated herein by reference).

Replacement of an amido linkage in an anti-coreceptor binding peptide with a —CH₂-sulfonamide linkage can be achieved by reducing the carboxyl (—COOH) group of a suitably protected amino acid to the —CH₂OH group, and the hydroxyl group is then converted to a suitable leaving group such as a tosyl group by conventional methods. Reaction of the tosylated derivative with, for example, thioacetic acid followed by hydrolysis and oxidative chlorination will provide for the —CH₂—S(O)₂Cl functional group which replaces the carboxyl group of the otherwise suitably protected amino acid. Use of this suitably protected amino acid analogue in peptide synthesis provides for inclusion of an —CH₂S(O)₂NR— linkage which replaces the amido linkage in the peptide thereby providing a peptide mimetic. For a more complete description on the conversion of the carboxyl group of the amino acid to a —CH₂S(O)₂Cl group, see, e.g., Weinstein and Boris (Chemistry & Biochemistry of Amino Acids. Peptides and Proteins, Vol. 7, pp. 267-357, Marcel Dekker, Inc., New York, 1983, incorporated herein by reference). Replacement of an amido linkage in an anti-coreceptor binding peptide with a urea linkage can be achieved in the manner known to the skilled artisan.

Secondary amine linkages wherein a —CH₂NH— linkage replaces the amido linkage in the peptide can be prepared by employing, for example, a suitably protected dipeptide analogue wherein the carbonyl bond of the amido linkage has been reduced to a CH₂ group by conventional methods. For example, in the case of diglycine, reduction of the amide to the amine will yield after deprotection H₂NCH₂CH₂NHCH₂ COOH which is then used in N-protected form in the next coupling reaction. The preparation of such analogues by reduction of the carbonyl group of the amido linkage in the dipeptide is well known in the art.

The anti-coreceptor binding agents of the present invention may exist in a monomeric form with no disulfide bond formed with the thiol groups of the cysteine residue(s). Alternatively, an intermolecular disulfide bond between the thiol groups of cysteines on two or more peptides can be produced to yield a multimeric (e.g., dimeric, tetrameric or higher oligomeric) compound. Certain of these peptides can be cyclized or dimerized via displacement of the leaving group by the sulfur of a cysteine or homocysteine residue (see, e.g., Barker et al., J. Med. Chem. 35:2040-2048, 1992; and/or et al., J. Org. Chem. 56:3146-3149, 1991, each incorporated herein by reference). Thus, one or more native cysteine residues may be substituted with a homocysteine. Intramolecular or intermolecular disulfide derivatives of anti-coreceptor binding agents provide analogs in which one of the sulfurs has been replaced by a CH₂ group or other isostere for sulfur. These analogs can be made via an intramolecular or intermolecular displacement, using methods known in the art as shown below. One of skill in the art will readily appreciate that this displacement can also occur using other homologs of the a-amino-g-butyric acid derivative shown above and homocysteine.

All of the naturally occurring, recombinant, and synthetic peptides and peptide analogs and mimetics of the invention can be used for screening (e.g., in kits and/or screening assay methods) to identify additional compounds, including other peptides and peptide mimetics, that will function as anti-coreceptor binding agents within the methods and compositions of the invention. Several methods of automating assays have been developed in recent years so as to permit screening of tens of thousands of compounds in a short period (see, e.g., Fodor et al., Science 251:767-773, 1991, and U.S. Pat. Nos. 5,677,195; 5,885,837; 5,902,723; 6,027,880; 6,040,193; and 6,124,102, each incorporated herein by reference). For example, large combinatorial libraries of the compounds can be constructed by the encoded synthetic libraries (ESL) method described in, e.g., WO 95/12608, WO 93/06121, WO 94/08051, WO 95/35503, and WO 95/30642 (each incorporated by reference). Peptide libraries can also be generated by phage display methods (see, e.g., WO 91/18980, incorporated herein by reference). Many other publications describing chemical diversity libraries and screening methods are also considered reflective of the state of the art pertaining to these aspects of the invention and are generally incorporated herein.

In one general screening strategy within the invention, new agonists and antagonists against HIV-coreceptor binding can be readily identified using the anti-coreceptor binding peptides of the invention incorporated within highly automated assay methods, e.g., using a purified chemokine receptor. Of particular importance are antagonist compounds that have multi-tropic or multi-specific binding affinity or activity, i.e., which inhibit or block HIV interactions with distinct (e.g., both CXCR4 and CCR5) coreceptors and thereby inhibit or prevent infection by both T cell tropic (lymphotropic) and macrophage tropic (m-tropic) HIV strains.

One method of screening for new anti-coreceptor binding agents (e.g., small molecule drug peptide mimetics) utilizes eukaryotic or prokaryotic host cells which are stably transformed with recombinant DNA molecules expressing an anti-coreceptor binding peptide. Such cells, either in viable or fixed form, can be used for standard ligand/receptor binding assays (see, e.g., Parce et al., Science 246:243-247, 1989; and Owicki et al., Proc. Natl. Acad. Sci. USA 87:4007-4011, 1990, each incorporated herein by reference). Competitive assays are particularly useful, where the cells are contacted and incubated with a labeled receptor or antibody having known binding affinity to the peptide ligand, and a test compound or sample whose binding affinity is being measured. The bound and free labeled binding components are then separated to assess the degree of ligand binding. The amount of test compound bound is inversely proportional to the amount of labeled receptor binding to the known source. Any one of numerous techniques can be used to separate bound from free ligand to assess the degree of ligand binding. This separation step can involve a conventional procedure such as adhesion to filters followed by washing, adhesion to plastic followed by washing, or centrifugation of the cell membranes. Viable cells can also be used to screen for the effects of drugs on coreceptor-mediated functions and biological activities, e.g., HIV viral fusion, cell entry, replication, and the like. Some detection methods allow for elimination of a separation step, e.g., a proximity sensitive detection system.

Another technique for drug screening within the invention involves an approach which provides high throughput screening for compounds having suitable binding affinity to a target molecule, e.g., a chemokine receptor, and is described in detail in European Patent Application 84/03564, published on Sep. 13, 1984. First, large numbers of different test compounds, e.g., small peptides, are synthesized on a solid substrate, e.g., plastic pins or some other appropriate surface, (see, e.g., Fodor et al., Science 251:767-773, 1991, and U.S. Pat. Nos. 5,677,195; 5,885,837; 5,902,723; 6,027,880; 6,040,193; and 6,124,102, each incorporated herein by reference). Then all of the pins are reacted with a solubilized anti-coreceptor binding agent of the invention, and washed. The next step involves detecting bound anti-coreceptor binding agent.

Rational drug design may also be based upon structural studies of the molecular shapes of the anti-coreceptor binding agents. Various methods are available and well known in the art for characterizing, mapping, translating, and reproducing structural features of anti-coreceptor binding agents to guide the production and selection of new anti-coreceptor binding mimetics, including for example x-ray crystallography and 2 dimensional NMR techniques. These and other methods, for example, will allow reasoned prediction of which amino acid residues present in a selected anti-coreceptor binding peptide forms molecular contact regions necessary for peptide-coreceptor binding and specificity (see, e.g., Blundell and Johnson, Protein Crystallography, Academic Press, N.Y., 1976, incorporated herein by reference).

Operable anti-coreceptor binding analogs and mimetics within the invention retain partial, complete or enhanced activity compared to native anti-coreceptor binding peptides, for example, partial or complete activity for inhibiting HIV-coreceptor binding, HIV viral fusion, cell entry, and/or replication, or HIV-related disease occurrence or progression. In this regard, operable anti-coreceptor binding analogs and mimetics for use within the invention will retain at least 50%, often 75%, and up to 95-100% or greater levels of one or more selected activities as compared to the same activity observed for a selected native HIV-1 gp120 anti-coreceptor binding peptide. These biological properties of altered peptides or non-peptide mimetics can be determined according to any suitable assay disclosed or incorporated herein, for example, by determining the ability of an anti-coreceptor binding analog or mimetic to inhibit HIV viral fusion to coreceptor positive target cells.

In accordance with the description herein, the compounds of the invention are useful in vitro as unique tools for analyzing the nature and function of gp120 interactions with chemokine receptors, and will also serve as leads in various programs for designing additional peptide and non-peptide (e.g., small molecule drug) inhibitors of HIV-1.

In addition, the anti-coreceptor binding peptides and peptide analogs of the invention are useful as immunogens, or components of immunogens, for eliciting an immune response in mammalian subjects, for example, to provide a protective immune response to prevent or treat HIV infection. In this aspect, the peptides of the invention can be administered alone or in a formulation comprising the peptide and a pharmaceutically acceptable carrier or adjuvant, with or without additional active or inactive ingredients such immune modulatory agents (e.g., cytokines). In certain embodiments, the peptides are administered as immunogens in the form of a conjugate (e.g., a multimeric peptide, or a peptide/carrier or peptide/hapten conjugate). In one embodiment, a peptide is conjugated with a multimerizing polypeptide as described above. Alternatively, a multimeric construct of immunogenic peptides, for example, comprising repeat peptide subunits, or containing two or more different peptides, can be employed, which contain one or multiple immunogenic epitope(s) that elicit a specific, humoral and/or cell-mediated (e.g. CTL) immune response directed against the immunizing peptide(s).

Typically, the immune response will be marked by production of antibodies that bind the immunizing peptide(s) or peptide conjugate(s) with high affinity or avidity, but do not similarly recognize unrelated peptides. In other embodiments, the antibodies recognize the immunizing peptide(s) or peptide conjugate(s) but fail to bind with high affinity or avidity to chemokines, or to peptides derived from chemokines. In yet additional embodiments, antibodies generated against the anti-coreceptor binding peptide(s) or conjugate(s) bind to gp120 only when the gp120 protein is in a “bound” or “activated” state. For example, when gp120 is bound to a CD4 receptor this may result in a conformational change or other activation event that exposes a gp120 coreceptor binding domain. Antibodies that specifically target gp120 during this vulnerable activation period, (e.g., by recognizing one or more “masked” or “hidden” epitopes of gp120 that are not exposed for immune targeting when gp120 is in a “free” conformation or activity state, are particularly useful within the invention.

Thus, the invention also provides diagnostic and therapeutic antibodies, including monoclonal antibodies, and related compositions and methods for use in the management and treatment of HIV infection and related disease. The antibodies specifically recognize anti-coreceptor binding peptides of the invention and are therefore useful for blocking HIV-coreceptor interactions when administered in vivo. For example, monoclonal antibodies can be generated that specifically bind the anti-coreceptor binding peptides of the invention, which antibodies can be purified and administered to a patient to block or inhibit HIV-coreceptor interactions, including binding or “docking,” and HIV viral fusion, entry, replication and HIV-related disease occurrence or progression. These activities are typically mediated, at least in part, by in vivo binding of the antibodies to gp120 (and therefore to intact HIV virus), at least in the protein's activated state upon binding to CD4. These immunotherapeutic reagents often include humanized antibodies, and can be combined for therapeutic use with additional active or inert ingredients as disclosed herein, e.g., in conventional pharmaceutically acceptable carriers or diluents, e.g., immunogenic adjuvants, and optionally with adjunctive or combinatorially active agents such as antiretroviral drugs.

The production of non-human monoclonal antibodies, e.g., murine or rat, can be accomplished by, for example, immunizing the animal with a preparation comprising purified peptides of the invention, or purified gp120. The immunogen, often comprising a peptide/hapten complex or other conjugate as described herein, can be obtained from a natural source, by peptides synthesis or preferably by recombinant expression. Antibody-producing cells obtained from the immunized animals are immortalized and screened for the production of an antibody which binds to gp120 or a specific anti-coreceptor binding peptide (see, e.g., Harlow & Lane, Antibodies, A Laboratory Manual, Cold Springs Harbor Press, Cold Spring Harbor, N.Y., 1988, incorporated by reference for all purposes).

Humanized forms of mouse antibodies can be generated by linking the CDR regions of non-human antibodies to human constant regions by recombinant DNA techniques (see, e.g., Queen et al., Proc. Natl. Acad. Sci. USA 86:10029-10033, 1989 and WO 90/07861, each incorporated by reference). Human antibodies can be obtained using phage-display methods (see, e.g., WO 91/17271; WO 92/01047, each incorporated herein by reference). In these methods, libraries of phage are produced in which members display different antibodies on their outer surfaces. Antibodies are usually displayed as Fv or Fab fragments. Phage displaying antibodies with a desired specificity are selected by affinity enrichment to human cytochrome P450 or a fragment thereof. Human antibodies are selected by competitive binding experiments, or otherwise, to have the same epitope specificity as a particular mouse antibody.

The invention further provides fragments of the intact antibodies described above. Typically, these fragments compete with the intact antibody from which they were derived for specific binding to anti-coreceptor binding peptides and/or gp120. Antibody fragments include separate heavy chains, light chains Fab, Fab′ F(ab′)₂, Fv, and single chain antibodies. Fragments can be produced by enzymic or chemical separation of intact immunoglobulins. For example, a F(ab′)₂ fragment can be obtained from an IgG molecule by proteolytic digestion with pepsin at pH 3.0-3.5 using standard methods such as those described in Harlow and Lane, supra. Fab fragments may be obtained from F(ab′)₂ fragments by limited reduction, or from whole antibody by digestion with papain in the presence of reducing agents. Fragments can also be produced by recombinant DNA techniques. Segments of nucleic acids encoding selected fragments are produced by digestion of full-length coding sequences with restriction enzymes, or by de novo synthesis. Often fragments are expressed in the form of phage-coat fusion proteins. This manner of expression is advantageous for affinity-sharpening of antibodies.

To produce antibodies of the invention recombinantly, nucleic acids encoding light and heavy chain variable regions, optionally linked to constant regions, are inserted into expression vectors. The light and heavy chains can be cloned in the same or different expression vectors. The DNA segments encoding antibody chains are operably linked to control sequences in the expression vector(s) that ensure the expression of antibody chains. Such control sequences include a signal sequence, a promoter, an enhancer, and a transcription termination sequence. Expression vectors are typically replicable in the host organisms either as episomes or as an integral part of the host chromosome. E. coli is one prokaryotic host particularly useful for expressing antibodies of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which typically contain expression control sequences compatible with the host cell (e.g., an origin of replication) and regulatory sequences such as a lactose promoter system, a tryptophan (trp) promoter system, a beta-lactamase promoter system, or a promoter system from phage lambda. Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

Mammalian tissue cell culture can also be used to express and produce the antibodies of the present invention (see, e.g., Winnacker, From Genes to Clones, VCH Publishers, N.Y., 1987, incorporated herein by reference). Eukaryotic cells are preferred, because a number of suitable host cell lines capable of secreting intact antibodies have been developed. Preferred suitable host cells for expressing nucleic acids encoding the immunoglobulins of the invention include: monkey kidney CV1 line transformed by SV40 (COS-7, ATCC CRL 1651); human embryonic kidney line (293) (Graham et al., J. Gen. Virol. 36:59, 1977, incorporated herein by reference); baby hamster kidney cells (BHK, ATCC CCL 10); Chinese hamster ovary-cells-DHFR (CHO, Urlaub and Chasin, Proc. Natl. Acad. Sci. USA 77:4216, 1980, incorporated herein by reference); mouse sertoli cells (TM4, Mather, Biol. Reprod. 23:243-251, 1980, incorporated herein by reference); monkey kidney cells (CVI ATCC CCL 70); African green monkey kidney cells (VERO-76, ATCC CRL 1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); and, TRI cells (Mather et al., Annals N.Y. Acad. Sci. 383:44-46, 1982, incorporated herein by reference); and baculovirus cells.

The vectors containing the polynucleotide sequences of interest (e.g., the heavy and light chain encoding sequences and expression control sequences) can be transferred into the host cell. Calcium chloride transfection is commonly utilized for prokaryotic cells, whereas calcium phosphate treatment or electroporation can be used for other cellular hosts (see, e.g., Sambrook et al., Molecular Cloning: A Laboratory Manual, 2nd ed., Cold Spring Harbor Press, Cold Spring Harbor, N.Y., 1989, incorporated herein by reference). When heavy and light chains are cloned on separate expression vectors, the vectors are co-transfected to obtain expression and assembly of intact immunoglobulins. After introduction of recombinant DNA, cell lines expressing immunoglobulin products are cell selected. Cell lines capable of stable expression are preferred (i.e., undiminished levels of expression after fifty passages of the cell line).

Once expressed, the whole antibodies, their dimers, individual light and heavy chains, or other immunoglobulin forms of the present invention can be purified according to standard procedures of the art, including ammonium sulfate precipitation, affinity columns, column chromatography, gel electrophoresis and the like (see, e.g., Scopes, Protein Purification, Springer-Verlag, N.Y., 1982, incorporated herein by reference). Substantially pure immunoglobulins of at least about 90 to 95% homogeneity are preferred, and 98 to 99% or more homogeneity most preferred.

The anti-coreceptor binding agents of the invention can also generally be used in drug screening compositions and procedures, as noted above, to identify additional compounds having binding affinity to chemokine receptors and/or act as agonists or antagonists to gp120-coreceptor or chemokine-chemokine receptor interactions and related biological activities. Various screening methods and formats are available and well known in the art. Subsequent biological assays can then be utilized to determine if the screened compound has intrinsic coreceptor binding, agonist or antagonist, or other desired activity useful within the invention. Thus, in one example, the anti-coreceptor binding agents of the invention are useful as competitive binding agents in assays to screen for new HIV coreceptor agonists and antagonists. In such assays, the compounds of the invention can be used without modification or can be modified in a variety of ways; for example, by labeling, such as covalently or non-covalently joining a moiety which directly or indirectly provides a detectable signal. Direct labeling moieties include, for example, radiolabels, enzymes such as peroxidase and alkaline phosphatase (see, e.g., U.S. Pat. No. 3,645,090; and U.S. Pat. No. 3,940,475, each incorporated herein by reference), and fluorescent labels. Moieties for indirect labeling include biotin and avidin, a binding pair that can be coupled to one constituent and the other to a label. The compounds may also include spacers or linkers in cases where the compounds are to be attached to a solid support.

The anti-coreceptor binding agents of the invention can also be employed, based on their ability to bind HIV coreceptors (chemokine receptors), as reagents for detecting and/or quantifying HIV coreceptors on living cells, fixed cells, in biological fluids, in tissue homogenates, in purified, natural biological materials, and the like. For example, by labeling such peptides, one can identify and/or quantify cells having HIV coreceptors on their surfaces. In addition, based on their ability to bind HIV coreceptors, the peptides of the present invention can be used in in situ staining, FACS (fluorescence-activated cell sorting), Western blotting, ELISA, and the like. Further, the peptides of the present invention can be used in receptor purification, or in purifying cells expressing HIV coreceptors.

The compounds of the present invention can also be utilized as commercial reagents for various medical research and diagnostic uses. Such uses include but are not limited to: (1) use as a calibration standard for quantitating the activities of candidate HIV-coreceptor binding agonists and antagonists in a variety of functional assays; (2) use in structural analysis of HIV coreceptors through co-crystallization; and (3) use to investigate the mechanism of HIV coreceptor binding and activation.

The present invention also provides reagents, formulations, kits, and methods which provide significant prophylactic and therapeutic values. In one embodiment of the invention, methods and compositions employ an anti-coreceptor binding agent for preventing and/or inhibiting HIV-1 binding to a host cell thereby ameliorating HIV, i.e., HIV-1 infection or a selected disease or condition associated therewith. Additional methods and compositions are provided to treat, prevent or delay the occurrence or AIDS or ARC. In yet additional embodiments, the methods and compositions of the invention can be used to treat other diseases and conditions which benefit from the compositions and methodologies disclosed herein, for example, a specific HIV-related disease or condition, such as Kaposi's sarcoma or an opportunistic viral (e.g., herpes) or bacterial (e.g., pneumonia) infection or condition.

In accordance with the various treatment methods of the invention, the anti-coreceptor binding agent is delivered to a patient or other subject in a manner consistent with conventional methodologies associated with management of the disorder for which treatment or prevention is sought. In accordance with the disclosure herein, a prophylactically or therapeutically effective amount of an anti-coreceptor binding agent is administered to a subject in need of such treatment for a time and under conditions sufficient to prevent and/or inhibit HIV binding to a host cell thereby preventing and/or inhibiting HIV infection and ameliorating a selected disease or condition. The term “subject” as used herein means any mammalian patient to which the compositions of the invention may be administered. Typical subjects intended for treatment with the compositions and methods of the present invention include humans, as well as non-human primates and other animals. Alternate subjects for administration of anti-coreceptor binding agents of the invention (either for a diagnostic, analytic, disease management or therapeutic purpose) include cells, cell explants, tissues and organs, particularly those originating from mammalian subjects at risk of developing or presently suffering from HIV infection.

To identify subject patients for prophylaxis or treatment according to the methods of the invention, accepted screening methods are employed to determine risk factors associated with HIV infection, or to determine the status of an existing HIV infection or related condition in a subject. These screening methods include, for example, conventional work-ups to determine sexual and drug-use related risk factors, as well as diagnostic methods such as various ELISA immunoassay methods, which are available and well known in the art to detect and/or characterize HIV infection and related disease. These and other routine methods allow the clinician to select patients in need of therapy using the anti-coreceptor binding agents of the invention. In accordance with these methods and principles, anti-coreceptor binding agent therapy can be implemented as an independent prophylaxis or treatment program or as a follow-up, adjunct or coordinate treatment regimen to other treatments, for example other anti-HIV treatments such as drug therapy (AZT, DDI, protease inhibitors and other anti-retroviral drugs), surgery, vaccination, immunotherapy, hormone treatment, cell, tissue, or organ transplants, and the like.

Within the compositions and methods of the invention, the anti-coreceptor binding agent is typically formulated with a pharmaceutically acceptable carrier and administered in an amount sufficient to inhibit virus binding and initiation or progression of HIV infection or a related disease or condition in the subject. According to the methods of the invention, the anti-coreceptor binding agent can be administered to subjects by a variety of administration modes, including by intramuscular, subcutaneous, intravenous, intra-atrial, intra-articular, intraperitoneal, parenteral, oral, rectal, intranasal, intrapulmonary, transdermal or topically to the eyes, ears, skin or mucosal surfaces. Alternatively, the anti-coreceptor binding agent may be administered ex vivo by direct exposure to cells, tissues or organs originating from a mammalian subject, for example, as a component of an ex vivo tissue or organ treatment formulation that contains the anti-coreceptor binding agent in a biologically suitable, liquid or solid carrier.

For prophylactic and treatment purposes, the anti-coreceptor binding agent can be administered to the subject in a single bolus delivery, via continuous delivery (e.g., continuous intravenous or transdermal delivery) over an extended time period, or in a repeated administration protocol (e.g., on an hourly, daily or weekly basis). The various dosages and delivery protocols contemplated for administration of anti-coreceptor binding agents are therapeutically effective to inhibit the occurrence or alleviate one or more symptoms of HIV infection. An “anti-HIV therapeutically effective amount” of the anti-coreceptor binding agent thus refers to an amount that is effective, at dosages and for periods of time necessary, to achieve detectable inhibition of HIV binding and/or infection (initiation or progression) or a related condition (e.g., in HIV-exposed versus unexposed, or treated versus untreated, test and control subjects). In certain embodiments, a therapeutically effective amount of the anti-coreceptor binding agent, depending on the selected mode, frequency and duration of administration, will be effective to reduce or prevent HIV binding and infection of cells of the patient. Alternatively or in addition to these effects, a therapeutically effective dosage of the anti-coreceptor binding agent, which can include repeated doses within an prolonged prophylaxis or treatment regimen, will alleviate one or more symptoms or detectable conditions associated with HIV infection. Determination of effective dosages in this context is typically based on animal model studies followed up by human clinical trials and is guided by determining effective dosages and administration protocols that significantly reduce the occurrence or severity of HIV infection or related disease symptoms or conditions in the subject, which may be any of a range of accepted, e.g., murine or non-human primate, animal model subjects known in the art. Alternatively, effective dosages can be determined using in vitro models (e.g., immunologic and histopathologic assays). Using such models, only ordinary calculations and adjustments are typically required to determine an appropriate concentration and dose to administer an effective amount of anti-coreceptor binding agent (e.g., intranasally effective, transdermally effective, intravenously effective, or intramuscularly effective) to a subject to elicit a desired response. In alternative embodiments, an “effective amount” or “effective dose” of the anti-coreceptor binding agent may simply inhibit one or more selected biological activity(ies) correlated with HIV-coreceptor binding, as set forth above.

The actual dosage of anti-coreceptor binding agent will of course vary according to factors such as the risk or state of infection or disease, the subject's age, and weight, as well as the established potency of the anti-coreceptor binding agent for eliciting the desired activity or biological response in the subject. Dosage regimens can be adjusted to provide an optimum prophylactic therapeutic response. A therapeutically effective amount is also one in which any toxic or detrimental side effects of the anti-coreceptor binding agent is outweighed by therapeutically beneficial effects. A non-limiting range for a therapeutically effective amount of the anti-coreceptor binding agent 0.01 μg/kg-10 mg/kg, more typically between about 0.05 and 5 mg/kg, and in certain embodiments between about 0.2 and 2 mg/kg. Dosages within this range can be achieved by single or multiple administrations, including, e.g., multiple administrations per day, daily or weekly administrations. Per administration, it is desirable to administer at least one microgram of anti-coreceptor binding agent, more typically between about 10 μg and 5.0 mg, and in certain embodiments between about 100 μg and 1.0 or 2.0 mg to an average human subject. It is to be further noted that for each particular subject, specific dosage regimens should be evaluated and adjusted over time according to the individual need and professional judgment of the person administering or supervising the administration of the anti-coreceptor binding agent compositions.

Dosage of the anti-coreceptor binding agent can be varied by the attending clinician to maintain a desired concentration at the target site. For example, if an intravenous mode of delivery is selected local concentration of the anti-coreceptor binding agent in the bloodstream at a selected target tissue (e.g., circulating blood) can be between about 1-50 nanomoles of anti-coreceptor binding agent per liter, sometimes between about 1.0 nanomelia per liter and 10, 15 or 25 nanomoles per liter, depending on the subject's status and projected or measured response. Higher or lower concentrations can be selected based on the mode of delivery, e.g., trans-epidermal delivery versus delivery to a mucosal surface. Dosage should also be adjusted based on the release rate of the administered formulation, e.g., nasal spray versus powder, sustained release oral versus injected particle or transdermal delivery formulations, and the like. To achieve the same serum concentration level, for example, slow-release particles with a release rate of 5 nanomolar (under standard conditions) would be administered at about twice the dosage of particles with a release rate of 10 nanomolar.

Anti-coreceptor binding agents comprising HIV-1 gp120 peptides and peptide analogs can be readily constructed using peptide synthetic techniques, such as solid phase peptide synthesis (Merrifleld synthesis), and the like, or by recombinant DNA techniques, that are well known in the art. Techniques for making substitution mutations at predetermined sites in DNA include for example M13 mutagenesis. Manipulation of DNA sequences to produce substitutional, insertional, or deletional variants are conveniently described elsewhere such as Sambrook et al., (Molecular Cloning: A Laboratory Manual, 2nd ed., Cold Spring Harbor Laboratories, Cold Spring Harbor, N.Y., 1989). In accordance with these and related teachings, defined mutations can be introduced into a native gp120 peptide to generate analogs of interest by a variety of conventional techniques, e.g., site-directed mutagenesis of a cDNA copy of a portion of the gp120 gene encoding a selected peptide fragment, domain or motif. This can be achieved through and intermediate of single-stranded form, such as using the MUTA-gen® kit of Bio-Rad Laboratories (Richmond, Calif.), or a method using the double-stranded plasmid directly as a template such as the Chameleon® mutagenesis kit of Strategene (La Jolla, Calif.), or by the polymerase chain reaction employing either an oligonucleotide primer or a template which contains the mutation(s) of interest. A mutated subfragment can then be assembled into a complete anti-coreceptor binding peptide analog-encoding cDNA. A variety of other mutagenesis techniques are known and can be routinely adapted for use in producing the mutations of interest in a anti-coreceptor binding peptide analog-encoding cDNA and corresponding peptide analog of the invention.

In accordance with the present invention, anti-coreceptor binding agents are isolated and purified before administration to a subject so that contaminants are removed. In one method for obtaining purified anti-coreceptor binding peptides, a polynucleotide molecule, for example, a deoxyribonucleic acid (DNA) molecule, that defines a coding sequence for a selected anti-coreceptor binding peptide or peptide analog (e.g., a biologically active mutant or fragment of a peptide as disclosed herein bearing a deletion or substitution of 1, 2, 3 or more residues) is operably incorporated in a recombinant polynucleotide expression vector that direct expression of the peptide or analog in a suitable host cell. Exemplary methods for cloning and purifying anti-coreceptor binding peptides and analogs employing these novel polynucleotides and vectors are widely known in the art.

Briefly, a polynucleotide of the invention encoding an anti-coreceptor binding peptide or peptide analog is amplified by well known methods, such as the polymerase chain reaction (PCR). In this way the polynucleotide encoding the anti-coreceptor binding peptide, or a recombinantly modified version or portion thereof, is obtained for expression and purification according to conventional methods. A DNA vector molecule that incorporates a DNA sequence encoding the subject peptide or analog can be operatively assembled, e.g., by linkage using appropriate restriction fragments from various plasmids which are described elsewhere. Also contemplated by the present invention are ribonucleic acid (RNA) equivalents of the above described polynucleotides comprising a coding sequence for the subject anti-coreceptor binding peptide operatively linked in a polynucleotide expression construct for recombinant expression of the peptide or peptide analog.

Once a polynucleotide molecule encoding an anti-coreceptor binding peptide or analog is isolated and cloned, the peptide or analog can be expressed in a variety of recombinantly engineered cells. Numerous expression systems are available for expressing a DNA encoding anti-coreceptor binding peptide. The expression of natural or synthetic nucleic acids encoding an anti-coreceptor binding peptide is typically achieved by operably linking the DNA to a promoter (which is either constitutive or inducible) within an expression vector. By expression vector is meant a polynucleotide molecule, linear or circular, that comprises a segment encoding the anti-coreceptor binding peptide of interest, operably linked to additional segments that provide for its transcription. Such additional segments include promoter and terminator sequences. An expression vector also may include one or more origins of replication, one or more selectable markers, an enhancer, a polyadenylation signal, and the like. Expression vectors generally are derived from plasmid or viral DNA, and can contain elements of both. The term “operably linked” indicates that the segments are arranged so that they function in concert for their intended purposes, for example, transcription initiates in the promoter and proceeds through the coding segment to the terminator (see, e.g., Sambrook et al., Molecular Cloning: A Laboratory Manual, 2nd ed., Cold Spring Harbor Laboratories, Cold Spring Harbor, N.Y., 1989, incorporated herein by reference).

Expression vectors can be constructed which contain a promoter to direct transcription, a ribosome binding site, and a transcriptional terminator. Examples of regulatory regions suitable for this purpose in E. coli are the promoter and operator region of the E. coli tryptophan biosynthetic pathway as described by Yanofsky, (J. Bacteriol. 158:1018-1024, 1984, incorporated herein by reference) and the leftward promoter of phage lambda (Pk) as described by Herskowitz and Hagen, (Ann. Rev. Genet. 14:399-445, 1980, incorporated herein by reference). The inclusion of selection markers in DNA vectors transformed in E. coli is also useful. Examples of such markers include genes specifying resistance to ampicillin, tetracycline, or chloramphenicol. Vectors used for expressing foreign genes in bacterial hosts generally will contain a selectable marker, such as a gene for antibiotic resistance, and a promoter which functions in the host cell. Plasmids useful for transforming bacteria include pBR322 (Bolivar et al., Gene 2:95-113, 1977, incorporated herein by reference), the pUC plasmids (Messing, Meth. Enzymol. 101:20-77, 1983; Vieira and Messing, Gene 19:259-268. 1982, each incorporated herein by reference), pCQV2, and derivatives thereof Plasmids may contain both viral and bacterial elements.

A variety of prokaryotic expression systems can be used to express anti-coreceptor binding peptides and peptide analogs. Examples include E. coli, Bacillus, Streptomyces, and the like. Detection of the expressed peptide is achieved by methods such as radioimmunoassay, Western blotting techniques or immunoprecipitation. For expression in eukaryotes, host cells for use in practicing the invention include mammalian, avian, plant, insect, and fungal cells. Fungal cells, including species of yeast (e.g., Saccharomyces spp., Schizosaccharomyces spp.) or filamentous fungi (e.g., Aspergillus spp., Neurospora spp.) can be used as host cells within the present invention. Strains of the yeast Saccharomyces cerevisiae can be used. As explained briefly below, anti-coreceptor binding peptides and analogs can be expressed in these eukaryotic systems.

Suitable yeast vectors for use in the present invention include YRp7 (Struhl et al., Proc. Natl. Acad. Sci. USA 76:1035-1039, 1978, incorporated herein by reference), YEp13 (Broach et al., Gene 8:121-133, 1979, incorporated herein by reference), POT vectors (U.S. Pat. No. 4,931,373, incorporated herein by reference), pJDB249 and pJDB219 (Beggs, Nature 275:104-108, 1978, incorporated herein by reference) and derivatives thereof. Such vectors generally include a selectable marker, which can be one of any number of genes that exhibit a dominant phenotype for which a phenotypic assay exists to enable transformants to be selected. Often, the selectable marker will be one that complements host cell auxotrophy, provides antibiotic resistance and/or enables a cell to utilize specific carbon sources, for example, LEU2 (Broach et al., Gene 8:121-133, 1979), URA3 (Botstein et al., Gene 8:17, 1979, incorporated herein by reference), HIS3 (Struhl et al., Proc. Natl. Acad. Sci. USA 76:1035-1039, 1978) or POT1 (U.S. Pat. No. 4,931,373). Another suitable selectable marker available for use within the invention is the CAT gene, which confers chloramphenicol resistance on yeast cells.

Examples of promoters for use in yeast include promoters from yeast glycolytic genes (Hitzeman et al., J. Biol. Chem. 255:12073-12080, 1980; Alber and Kawasaki, J. Mol. Appl. Genet. 1:419434, 1982; U.S. Pat. No. 4,599,311) or alcohol dehydrogenase genes (Young et al., Genetic Engineering of Microorganisms for Chemicals, Hollaender et al., eds., p. 355, Plenum, N.Y., 1982; Ammerer, Meth. Enzymol. 101:192-201, 1983). The TPI1 promoter (U.S. Pat. No. 4,599,311) and the ADH2-4c promoter (Russell et al., Nature 304:652-654, 1983; and EP 284,044) also can be used. The expression units can also include a transcriptional terminator. An example of such a transcriptional terminator is the TPI1 terminator (Alber and Kawasaki, J. Mol. Appl. Genet. 1:419-434, 1982).

In addition to yeast, anti-coreceptor binding peptides and peptide analogs of the present invention can be expressed in filamentous fungi, for example, strains of the fungi Aspergillus (U.S. Pat. No. 4,935,349, which is incorporated herein by reference). Examples of useful promoters include those derived from Aspergillus nidulans glycolytic genes, such as the ADH3 promoter and the tpiA promoter. An example of a suitable terminator is the ADH3 terminator (McKnight et al., EMBO J. 4: 2093-2099, 1985, incorporated herein by reference). The expression units utilizing such components are cloned into vectors that are capable of insertion into the chromosomal DNA of Aspergillus.

Techniques for transforming fungi are well known in the literature, and have been described, for instance, by Beggs (Nature 275:104-108, 1978), Hinnen et al., (Proc. Natl. Acad. Sci. USA 75:1929-1933, 1978), Yelton et al., (Proc. Natl. Acad. Sci. USA 81:1740-1747, 1984), and Russell (Nature 301:167-169, 1983), each incorporated herein by reference. The genotype of the host cell generally contains a genetic defect that is complemented by the selectable marker present on the expression vector. Choice of a particular host and selectable marker is well within the level of ordinary skill in the art.

In addition to fungal cells, cultured mammalian cells can be used as host cells within the present invention. Examples of cultured mammalian cells for use in the present invention include the COS-1 (ATCC CRL 1650), BHK, and 293 (ATCC CRL 1573; Graham et al., J. Gen. Virol. 6:59-72, 1977, incorporated herein by reference) cell lines. An example of a BHK cell line is the BHK 570 cell line (deposited with the American Type Culture Collection under accession number CRL 10314). In addition, a number of other mammalian cell lines can be used within the present invention, including rat Hep I (ATCC CRL 600), rat Hep II (ATCC CRL 1548), TCMK (ATCC CCL 139), human lung (ATCC CCL 75.1), human hepatoma (ATCC HTB-52), Hep G2 (ATCC HB 8065), mouse liver (ATCC CCL 29.1), NCTC 1469 (ATCC CCL 9.1) and DUKX cells (Urlaub and Chasin, Proc. Natl. Acad. Sci USA 77:4216-4220, 1980, incorporated herein by reference).

Mammalian expression vectors for use in carrying out the present invention include a promoter capable of directing the transcription of a cloned cDNA. Either viral promoters or cellular promoters can be used. Viral promoters include the immediate early cytomegalovirus (CMV) promoter (Boshart et al., Cell 41:521-530, 1985, incorporated herein by reference) and the SV40 promoter (Subramani et al., Mol. Cell. Biol. 1:854-864, 1981, incorporated herein by reference). Cellular promoters include the mouse metallothionein-1 promoter (U.S. Pat. No. 4,579,821, incorporated herein by reference), a mouse V1 promoter (Bergman et al., Proc. Natl. Acad. Sci. USA 81:7041-7045, 1983; Grant et al., Nuc. Acids Res. 15:5496, 1987, each incorporated herein by reference), a mouse V1 promoter (Loh et al., Cell 33:85-93, 1983, incorporated herein by reference), and the major late promoter from Adenovirus 2 (Kaufman and Sharp, Mol. Cell. Biol. 2:1304-13199, 1982, incorporated herein by reference).

Cloned DNA sequences can be introduced into cultured mammalian cells by, for example, calcium phosphate-mediated transfection (Wigler et al., Cell 14:725, 1978; Corsaro and Pearson, Somatic Cell Genetics 7:603, 1981; Graham and Van der Eb, Virology 52:456, 1973; each incorporated by reference herein in their entirety). Other techniques for introducing cloned DNA sequences into mammalian cells can also be used, such as electroporation (Neumann et al., EMBO J. 1:841-845, 1982, incorporated herein by reference) or cationic lipid-mediated transfection (Hawley-Nelson et al., Focus 15:73-79, 1993, incorporated herein by reference) using, e.g., a 3:1 liposome formulation of 2,3-dioleyloxy-N-[2 (sperminecarboxyamido)ethyl]-N,N-dimethyl-1-propanaminiumtrifluoroacetate and dioleoly-phosphatidylethanolamine in water Lipofectamine reagent, GIBCO-BRL). To identify cells that have integrated the cloned DNA, a selectable marker is generally introduced into the cells along with the gene or cDNA of interest. Examples of selectable markers for use in cultured mammalian cells include genes that confer resistance to drugs, such as neomycin, hygromycin, and methotrexate. The selectable marker can be an amplifiable selectable marker, for example the DHFR gene. Additional selectable markers are reviewed by Thilly (Mammalian Cell Technology, Butterworth Publishers, Stoneham, Mass., which is incorporated herein by reference). The choice of selectable markers is well within the level of ordinary skill in the art.

Selectable markers can be introduced into the cell on a separate plasmid at the same time as the polynucleotide encoding the anti-coreceptor binding peptide, or they may be introduced on the same plasmid. If on the same plasmid, the selectable marker and the peptide-encoding polynucleotide can be under the control of different promoters or the same promoter. Constructs of this latter type are known in the art (for example, U.S. Pat. No. 4,713,339). It also can be advantageous to add additional DNA, known as “carrier DNA” to the mixture which is introduced into the cells.

Transfected mammalian cells are allowed to grow for a period of time, typically 1-2 days, to begin expressing the polynucleotide sequence(s) of interest. Drug selection is then applied to select for growth of cells that are expressing the selectable marker in a stable fashion. For cells that have been transfected with an amplifiable selectable marker the drug concentration is increased in a stepwise manner to select for increased copy number of the cloned sequences, thereby increasing expression levels.

Host cells containing polynucleotide constructs of the present invention are then cultured to produce anti-coreceptor binding peptide. The cells are cultured according to standard methods in a culture medium containing nutrients required for growth of the host cells. A variety of suitable media are known in the art and generally include a carbon source, a nitrogen source, essential amino acids, vitamins, minerals and growth factors. The growth medium generally selects for cells containing the DNA construct by, for example, drug selection or deficiency in an essential nutrient which is complemented by the selectable marker on the DNA construct or co-transfected with the DNA construct.

Recombinantly produced anti-coreceptor binding peptides and peptide analogs as described above can be purified by techniques well known to those of ordinary skill in the art. For example, recombinantly produced peptides can be directly expressed or expressed as fusion proteins. The proteins can then be purified by a combination of cell lysis (e.g., sonication) and affinity chromatography. For fusion products, subsequent digestion of the fusion protein with an appropriate proteolytic enzyme releases the desired peptide.

The phrase “substantially purified” when referring to anti-coreceptor binding peptides or peptide analogs (including peptide fusions with other peptides and/or proteins) of the present invention, means a composition which is essentially free of other cellular components with which the peptides or analogs are associated in a non-purified, e.g., native state or environment. Purified peptide is generally in a homogeneous state although it can be in either in a dry state or in an aqueous solution. Purity and homogeneity are typically determined using analytical chemistry techniques such as polyacrylamide gel electrophoresis or high performance liquid chromatography.

Generally, substantially purified anti-coreceptor binding peptide comprises more than 80% of all macromolecular species present in a preparation prior to admixture or formulation of the peptide with a pharmaceutical carrier, excipient, buffer, absorption enhancing agent, stabilizer, preservative, adjuvant or other co-ingredient. More typically, the peptide is purified to represent greater than 90% of all proteins present in a purified preparation. In specific embodiments, the peptide is purified to greater than 95% purity or may be essentially homogeneous wherein other macromolecular species are not detectable by conventional techniques.

The peptides and analogs of the present invention can be purified to substantial purity by standard techniques well known in the art. Useful purification methods include selective precipitation with such substances as ammonium sulfate; column chromatography; affinity methods, including immunopurification methods; and others. See, for instance, R. Scopes, Protein Purification: Principles and Practice, Springer-Verlag: N.Y., 1982, incorporated herein by reference. In general, anti-coreceptor binding peptides can be extracted from tissues or cell cultures that express the peptides and then immunoprecipitated, whereafter the peptides can be further purified by standard protein chemistry/chromatographic methods.

For therapeutic administration, anti-coreceptor binding peptides, analogs and mimetics of the invention are typically formulated with a pharmaceutically acceptable carrier. As used herein, “pharmaceutically acceptable carrier” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, subcutaneous, parenteral, oral, transmucosal or transdermal administration. Depending on the route of administration, the active compound may be coated in a material to protect the compound from the action of acids and other natural conditions which may inactivate the compound.

In preparing pharmaceutical compositions of the present invention, it may be desirable to modify the anti-coreceptor binding agent, or combine or conjugate the peptide or mimetic compound with other agents, to alter pharmacokinetics and biodistribution of the anti-coreceptor binding agent. A number of methods for altering pharmacokinetics and biodistribution are known to persons of ordinary skill in the art. Examples of such methods include protection of peptides, proteins or complexes thereof in vesicles composed of other proteins, lipids, carbohydrates, or synthetic polymers. For example, anti-coreceptor binding agents can be incorporated into liposomes in order to enhance pharmacokinetics and biodistribution characteristics. A variety of methods are available for preparing liposomes, as described in, e.g., Szoka et al., (Ann. Rev. Biophys. Bioeng. 2:467, 1980; U.S. Pat. Nos. 4,235,871; 4,501,728 and 4,837,028, each incorporated herein by reference). For use with liposome delivery, the anti-coreceptor binding agent is typically entrapped within the liposome, or lipid vesicle, or is bound to the outside of the vesicle. Several strategies have been devised to increase the effectiveness of liposome-mediated delivery by targeting liposomes to specific tissues and specific cell types. Liposome formulations, including those containing a cationic lipid, have been shown to be safe and well tolerated in human patients (Treat et al., J. Natl. Cancer Instit. 82:1706-1710, 1990, incorporated herein by reference).

The compositions of the invention may alternatively contain as pharmaceutically acceptable carriers, substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like.

For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95%, more typically 25% to 75% of active ingredient.

Therapeutic compositions for administering the anti-coreceptor binding agent can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

In certain embodiments of the invention, the anti-coreceptor binding agent is administered in a time release formulation, for example in a composition which includes a slow release polymer, or by depot injection. The active peptide, analog or mimetic can be prepared with carriers that will protect against rapid release, for example a controlled release vehicle such as implants, transdermal patches, or microencapsulated delivery system. Prolonged delivery of the anti-coreceptor binding agent, or a biologically active analog or mimetic thereof, in various compositions of the invention can be brought about by including in the composition agents that delay absorption, for example, aluminum monosterate hydrogels and gelatin. When controlled release formulations of anti-coreceptor binding agents is desired, controlled release binders suitable for use in accordance with the invention include any biocompatible controlled-release material which is inert to the active ingredient and which is capable of incorporating the anti-coreceptor binding agent. Numerous such materials are known in the art. Useful controlled-release binders are materials which are metabolized slowly under physiological conditions following their subcutaneous or intramuscular injection in mammals (i.e., in the presence of bodily fluids which exist there). Appropriate binders include but are not limited to biocompatible polymers and copolymers previously used in the art in sustained release formulations. Such biocompatible compounds are non-toxic and inert to surrounding tissues, e.g., following subcutaneous or intramuscular injection, and do not trigger significant adverse effects such as immune response, inflammation, or the like. They are metabolized into metabolic products which are also biocompatible and easily eliminated from the body.

For example, a polymeric matrix derived from copolymeric and homopolymeric polyesters having hydrolysable ester linkages may be used. A number of these are known in the art to be biodegradable and to lead to degradation products having no or low toxicity. Exemplary polymers include polyglycolic acids (PGA) and polylactic acids (PLA), poly(DL-lactic acid-co-glycolic acid)(DL PLGA), poly(D-lactic acid-coglycolic acid) (D PLGA) and poly(L-lactic acid-co-glycolic acid)(L PLGA). Other useful biodegradable or bioerodable polymers include but are not limited to such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels such as poly(hydroxyethyl methacrylate), polyamides, poly(amino acids) (i.e., L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides and copolymers thereof. Many methods for preparing such formulations are generally known to those skilled in the art (see, e.g., Sustained and Controlled Release Drug Delivery Systems, J. R. Robinson, ed., Marcel Dekker, Inc., New York, 1978, incorporated herein by reference). Useful formulations include controlled-release compositions such as are known in the art for the administration of leuprolide (trade name: Lupron.RTM.), e.g., microcapsules (U.S. Pat. Nos. 4,652,441 and 4,917,893, each incorporated herein by reference), injectable formulations (U.S. Pat. No. 4,849,228, incorporated herein by reference), lactic acid-glycolic acid copolymers useful in making microcapsules or injectable formulations (U.S. Pat. Nos. 4,677,191 and 4,728,721, each incorporated herein by reference), and sustained-release compositions for water-soluble peptides (U.S. Pat. No. 4,675,189, incorporated herein by reference). A long-term sustained release implant also may be used. These can be readily constructed to deliver therapeutic levels of the anti-coreceptor binding agent for at least 10 to 20 days, often at least 30 days, up to 60 days or longer. Long-term sustained release implants are well known to those of ordinary skill in the art and can incorporate some of the absorption delaying components described above. Such implants can be particularly useful by placing the implant near or directly within the target tissue or cell population, thereby affecting localized, high-doses of the anti-coreceptor binding agent at one or more sites of interest.

In alternate embodiments, anti-coreceptor binding agent may be delivered to a mucosal surface, e.g., orally, intranasally, or rectally, for prophylaxis or treatment of HIV infection and related disease. For mucosal administration, the peptide, analog or mimetic is typically combined with an inert diluent or an assimilable edible carrier. The anti-coreceptor binding agent may be enclosed in a hard or soft shell gelatin capsule, compressed into tablets, or incorporated directly into a subject's diet. For oral therapeutic administration, the anti-coreceptor binding agent may be incorporated with excipients and used in the form of ingestable tablets, buccal tablets, troches, capsules, elixirs, suspensions, syrups, wafers, and the like. Of course, taste-improving substances can be added in the case of oral administration forms. The percentage (e.g., by weight or by volume) of the anti-coreceptor binding agent in these compositions and preparations may, of course, be varied. As noted above, the amount of anti-coreceptor binding agent in such therapeutically useful compositions is generally such that a therapeutically effective dosage will be delivered.

For oral or rectal administration, the anti-coreceptor binding agent can be worked into tablets or other solid forms by being mixed with solid, pulverulent carrier substances, such as sodium citrate, calcium carbonate or dicalcium phosphate, and binders such as polyvinyl pyrrolidone, gelatin or cellulose derivatives, possibly by adding also lubricants such as magnesium stearate, sodium lauryl sulfate, “Carbowax” or polyethylene glycol. Solid delivery vehicles may contain a protein or peptide in a mixture with fillers, such as lactose, saccharose, mannitol, starches, such as potato starch or amylopectin, cellulose derivatives or highly dispersed silicic acids. In soft-gelatin capsules, the active substance is dissolved or suspended in suitable liquids, such as vegetable oils or liquid polyethylene glycols. As further forms, one can use plug capsules, e.g., of hard gelatin, as well as dosed soft-gelatin capsules comprising a softener or plasticizer, e.g., glycerin.

Alternatively, liquid dosage forms for delivering anti-coreceptor binding agents to mucosal surfaces include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms would also include minerals and other materials to make them compatible with the type of injection or delivery system chosen.

The therapeutic compositions of the invention typically must be sterile and stable under all conditions of manufacture, storage and use. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In certain embodiments of the invention, the anti-coreceptor binding agent is administered by topical delivery to a mucosal surface of the patient, for example, via intranasal or intrapulmonary delivery in the form of an aerosol spray or powder. According to one aspect of the invention, the anti-coreceptor binding agent is delivered in an intranasally or intrapulmonarily effective amount, typically in a selected volume of administered spray or powder, to achieve a desired therapeutic result. In related aspects of the invention, novel pharmaceutical compositions are provided for intranasal or intrapulmonary delivery that incorporate the anti-coreceptor binding agent in a powder or aqueous formulation. Intranasal or intrapulmonary administration allows self-administration of treatment by patients, provided that sufficient safeguards are in place to control and monitor dosing and side effects. Nasal or intrapulmonary administration also overcomes certain drawbacks of other administration forms, such as injections, that are painful and expose the patient to possible infections and may present drug bioavailability problems. Systems for aerosol dispensing of therapeutic liquids as a spray are well known. In one embodiment, metered doses of aerosolized anti-coreceptor binding agent are delivered by means of a specially constructed mechanical pump valve (See, for example, U.S. Pat. No. 4,511,069, incorporated herein by reference). This hand-held delivery device is uniquely nonvented so that sterility of the solution in the aerosol container is maintained indefinitely. Certain nasal and intrapulmonary spray solutions within the invention comprise an anti-coreceptor binding agent in a liquid carrier that optionally includes a nonionic surfactant for enhancing absorption of the drug and one or more buffers or other additives to minimize nasal or pulmonary irritation. In some embodiments, the nasal or pulmonary spray solution further comprises a propellant. Thus, in certain embodiments the pharmaceutical compositions comprising an anti-coreceptor binding agent administrable in fine particulate form (e.g., between about 0.5-5.0 μm, more typically between about 1.0-2.5 μm diameter particles) comprising a surfactant and propellant as pharmaceutically acceptable carriers (see, e.g., U.S. Pat. No. 5,902,789, incorporated herein by reference). The surfactant must be nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides, can be employed. Additional carrier can be included as desired, for example lecithin for intranasal delivery. The pH of the intranasal or intrapulmonary spray solution is typically between pH 6.8 and 7.2. Alternative means of mucosal administration for the anti-coreceptor binding agents of the invention may involve the use of powder carriers, for example ion exchange resins or adsorbent resin powders (see, e.g., U.S. Pat. No. 5,942,242, incorporated herein by reference).

In more detailed aspects of the invention, the anti-coreceptor binding agent is stabilized to extend its effective half-life following delivery to the subject, particularly for extending metabolic persistence in an active state within the physiological environment (e.g., in the bloodstream, at a mucosal surface, or within a connective tissue compartment or fluid-filled body cavity). For this purpose, the anti-coreceptor binding agent can be modified by chemical means, e.g., chemical conjugation, N-terminal capping, PEGylation, or recombinant means, e.g., site-directed mutagenesis or construction of fusion proteins, or formulated with various stabilizing agents or carriers. Thus stabilized, the anti-coreceptor binding agent administered as above retains anti-coreceptor activity for an extended period (e.g., 2 to 3, up to 5 to 10 fold greater stability) under physiological conditions compared to its non-stabilized form.

Numerous reports in the literature describe the potential advantages of pegylated proteins, which include their increased resistance to proteolytic degradation, increased plasma half-life, increased solubility and decreased antigenicity and immunogenicity (Nucci et al., Advanced Drug Deliver Reviews 6:133-155, 1991; Lu et al., Int. J. Peptide Protein Res. 43:127-138, 1994, each incorporated herein by reference). A number of proteins, including L-asparaginase, strepto kinase, insulin, and interleukin-2 have been conjugated to a poly(ethyleneglycol) (PEG) and evaluated for their altered biochemical properties as therapeutics (see, e.g., Ho et al., Drug Metabolism and Disposition 14:349-352, 1986; Abuchowski et al., Prep. Biochem. 9:205-211, 1979; and Rajagopaian et al., J. Clin. Invest. 75:413-419, 1985, each incorporated herein by reference). Although the in vitro biological activities of pegylated proteins may be decreased, this loss in activity is usually offset by the increased in vivo half-life in the bloodstream (Nucci, et al., Advanced Drug Deliver Reviews 6:133-155, 1991, incorporated herein by reference).

Several procedures have been reported for the attachment of PEG to proteins and peptides and their subsequent purification (Abuchowski et al., J. Biol. Chem. 252:3582-3586,1977; and Beauchamp et al., Anal. Biochem, 131:25-33, 1983, each incorporated herein by reference). Lu et al., (Int. J. Peptide Protein Res. 43:127-138, 1994) describe various technical considerations and compare PEGylation procedures for proteins versus peptides (see also, Katre et al., Proc. Natl. Acad. Sci. USA 84:1487-1491, 1987; Becker et al., Makromol. Chem. Rapid Commun. 3:217-223, 1982; Mutter et al., Makromol. Chem. Rapid Commun. 13:151-157, 1992; Merrifield, R. B., J. Am. Chem. Soc. 85:2149-2154, 1993; Lu et al., Peptide Res. 6:142-146, 1993; Lee et al., Bioconiugate Chem. 10:973-981, 1999; Nucci et al., Adv. Drug Deliv. Rev. 6:133-151, 1991; Francis et al., J. Drug Targeting 3:321-340, 1996; Zalipsky, Bioconjugate Chem. 6:150-165, 1995; Clark et al., J. Biol. Chem. 271:21969-21977, 1996; Pettit et al., J. Biol. Chem. 272:2312-2318, 1997; Delgado et al., Br. J. Cancer 73:175-182, 1996; Benhar et al., Bioconjugate Chem. 5:321-326, 1994; Benhar et al., J. Biol. Chem. 269:13398-13404, 1994; Wang et al., Cancer Res. 53:4588-4594, 1993; Kinstler et al., Pharm. Res. 13:996-1002, 1996, Filpula et al., Exp. Opin. Ther. Patents 2:231-245, 1999; Pelegrin et al., Hum. Gene Ther. 9:2165-2175, 1998, each incorporated herein by reference). Following these and other teachings in the art, the conjugation of anti-coreceptor binding peptides and analogs with poly(ethyleneglycol) polymers, is readily undertaken with the expected result of prolonging circulating life and/or reducing immunogenicity while maintaining an acceptable level of activity of the PEGylated anti-coreceptor binding agent.

Amine-reactive PEG polymers for use within the invention include SC-PEG with molecular masses of 2000, 5000, 10000, 12000, and 20000; U-PEG-10000; NHS-PEG-3400-biotin; T-PEG-5000; T-PEG-12000; and TPC-PEG-5000. Chemical conjugation chemistries for these polymers have been published (see, e.g., Zalipsky, Bioconjugate Chem. 6:150-165, 1995; Greenwald et al., Bioconjugate Chem. 7:638-641, 1996; Martinez et al., Macromol. Chem. Phys. 198:2489-2498, 1997; Hermanson, Bioconjugate Techniques, pp. 605-618, 1996; Whitlow et al., Protein Eng. 6:989-995, 1993; Habeeb, Anal. Biochem. 14:328-336, 1966; Zalipsky et al., Poly(ethyleneglycol) Chemistry and Biological Applications, pp. 318-341, 1997; Harlow et al., Antibodies: A Laboratory Manual pp. 553-612, Cold Spring harbor Laboratory, Plainview, N.Y., 1988; Milenic et al., Cancer Res. 51:6363-6371, 1991; Friguet et al., J. Immunol. Methods 77:305-319, 1985, each incorporated herein by reference). While phosphate buffers are commonly employed in these protocols, the choice of borate buffers may beneficially influence the PEGylation reaction rates and resulting products.

PEGylation of anti-coreceptor binding peptides and analogs may be achieved by modification of carboxyl sites (e.g., aspartic acid or glutamic acid groups in addition to the carboxyl terminus). The utility of PEG-hydrazide in selective modification of carbodimide-activated protein carboxyl groups under acidic conditions has been described (Zalipsky, Bioconjugate Chem. 6:150-165, 1995; Zalipsky et al., Poly(ethyleneglycol) Chemistry and Biological Applications, pp.318-341, American Chemical Society, Washington, D.C., 1997, each incorporated herein by reference). Alternatively, bifunctional PEG modification of anti-coreceptor binding peptides can be employed. In some procedures, charged amino acid residues, including lysine, aspartic acid, and glutamic acid, have a marked tendency to be solvent accessible on protein surfaces. Conjugation to carboxylic acid groups of proteins is a less frequently explored approach for production of protein bioconjugates. However, the hydrazide/EDC chemistry described by Zalipsky et al., (Zalipsky, Bioconjugate Chem. 6:150-165, 1995; Zalipsky et al., Poly(ethyleneglycol) Chemistry and Biological Applications, pp. 318-341, American Chemical Society, Washington, D.C., 1997, each incorporated herein by reference) offers a practical method of linking PEG polymers to protein carboxylic sites. For example, this alternate conjugation chemistry has been shown to be superior to amine linkages for PEGylation of brain-derived neurotrophic factor (BDNF) while retaining biological activity (Wu et al., Proc. Natl. Acad. Sci. U.S.A. 96:254-259, 1999, incorporated herein by reference). Maeda and colleagues have also found carboxyl-targeted PEGylation to be the preferred approach for bilirubin oxidase conjugations (Maeda et al., Poly(ethyleneglycol) Chemistry, Biotechnical and Biomedical Applications, J. M. Harris, ed., pp. 153-169, Plenum Press, New York, 1992, incorporated herein by reference).

In addition to PEGylation, anti-coreceptor binding agents of the invention can be modified to enhance circulating half-life by shielding the peptide or analog via conjugation to other known protecting or stabilizing compounds, or by the creation of fusion proteins with the peptide or analog linked to one or more carrier proteins, such as one or more immunoglobulin chains (see, e.g., U.S. Pat. Nos. 5,750,375; 5,843,725; 5,567,584 and 6,018,026, each incorporated herein by reference). These modifications will decrease the degradation, sequestration or clearance of the anti-coreceptor binding agent and result in a longer half-life in a physiological environment (e.g., in the circulatory system, or at a mucosal surface). The anti-coreceptor binding agents modified by PEGylation and other stabilizing methods are therefore useful with enhanced efficacy within the methods of the invention. In particular, the anti-coreceptor binding agents thus modified maintain activity for greater periods at a target site of delivery compared to the unmodified peptide or analog. Even when the anti-coreceptor binding agents are thus modified, they retain substantial biological activity for inhibiting HIV-coreceptor interactions and HIV infection and related disease.

In yet additional aspects of the invention, an anti-coreceptor binding agent is administered according to the foregoing methods in a coordinate therapy protocol with one or more additional anti-HIV agents or treatment steps. Thus, in various embodiments an anti-coreceptor binding agent is administered coordinately with one or more adjunct or combinatorially (e.g., additively or synergistically) effective treatment agents selected from anti-retroviral drugs (e.g., nucleoside reverse transcriptase inhibitors such as AZT (zidovudine), Videx® (ddI or didanosine), or Hivid® (ddC or zalcitabine); antiviral drugs (e.g., acyclovir); antibiotics (e.g., penicillins, cephalosporins, macrolides and lincosamides such as erythromycin, gentamycin, ceftriaxone, cefixime, azithromycin, spectinomycin, ofloxacin, ciprofloxacin, cefoxitin, clindamycin, metronidazole, amoxicillin, tetracycline, doxycycline); immunomodulatory agents (e.g., interferons (IFNα, IFNβ, IFNγ), interleukins (such as IL-1 or IL-2); G-CSF, GM-CSF, levamisole) particularly interferon); or vaccine agents. Other immune modulatory agents that are useful for coordinate administration with anti-coreceptor binding agents include corticosteroids, cytotoxic drugs, T-cell specific inhibitors, antisera, replacement immune globulins, and monoclonal antibodies. At times, specific immunosuppressive drugs can be used in conjunction with anti-coreceptor binding agent therapy, for example, azathioprine, cyclophosphamide, or cyclosprine. Some cytotoxic drugs may also be used for coordinate administration with anti-coreceptor binding agents, for example azathioprine, azathioprine sodium, chlorambucil, cyclophosphamide, methotrexate, methotrexate sodium, and/or clyclosprorine.

Additional combinatorial or adjunctive therapeutic agents for use within coordinate formulations and treatment methods of the invention may comprise a glucocorticoid or non-steroidal anti-inflammatory drug (NSAID). Glucocorticoids useful within this aspect of the invention include short-acting glucocorticoids (e.g., cortisone and hydrocortisone), intermediate-acting glucocorticoids (e.g., prednisone, prednisolone, meprednisone, methylprednisolone and triamcinolone), and long-acting glucocorticoids (betamethasone, dexamethasone and paramethasone). NSAID's useful within the invention include, aspirin, salicylates, naproxen, indomethacin, piroxicam, oxaprozin, phenylbutazone, ibuprofen, flurbiprofen, fenoprofen, and ketoprofen, and the like. Additional therapeutic agents for use in conjunction with anti-coreceptor binding agent therapy may include the anti-malarial drug hydroxychloroquine, or sulfasalazine. Also useful in coordinate therapy protocols with anti-coreceptor binding agents are antihistamines, including amino alkyl ethers (e.g., diphenhydramine, clemasine), ethylenediamines (e.g., pyrilamine, tripelennamine), alkylamines (e.g., brompheniramine, chlorpheniramine, dexchlorpheniramine, triprolidine), and phenothiazines (e.g., methdilazine, promethazine, trimeprazine).

Within alternate methods and compositions of the invention, the anti-coreceptor binding agent is administered as above coordinately, admixed or separately, simultaneously or sequentially, with of one or more of the foregoing “combinatorially effective or adjunct treatment agents”, in respective amounts sufficient (independently sufficient or combinatorially sufficient) to prevent or alleviate HIV infection or a disease condition or symptom associated therewith.

The instant invention also includes kits, packages and multicontainer units containing the above described pharmaceutical compositions, active ingredients, and/or means for administering the same for use in the prevention and treatment of HIV infection and related disease conditions. Briefly, these kits include a container or formulation which contains an anti-coreceptor binding agent, typically formulated in a pharmaceutical preparation with a biologically suitable carrier. The anti-coreceptor binding agent is optionally contained in a bulk dispensing container or unit or multi-unit dosage form. Optional dispensing means may be provided, for example, an intranasal or intrapulmonary spray applicator. Packaging materials optionally include a label or instruction which indicates that the pharmaceutical agent packaged therewith can be used for treating HIV and related disease conditions.

In more detailed embodiments of the invention, kits include reagents and/or devices for detecting the presence and/or status of HIV infection or related disease in a subject. For example, an immunological or molecular probe that binds or reacts with an HIV-specific marker detectable in blood or other biological samples to be obtained from the subject. Thus, the kits may contain ELISA probes for detecting HIV antigens, as well as additional, optional kit materials for collecting and/or processing samples for ELISA and other diagnostic assays. The kits may also contain suitable buffers, preservatives such as protease inhibitors, direct or sandwich-type labels for labeling probes, and/or developing reagents for detecting a signal from the label. Thus, a broad selection of therapeutic and diagnostic kits are provided within the invention based on the description herein, including kits that contain specific instructions for carrying out the prophylactic and treatment protocols and associated assays of the invention.

The invention is further illustrated by the following specific examples which are not intended in any way to limit the scope of the invention.

### EXAMPLES

Materials and Methods

The following examples describe computer modeling, synthesis and testing of co-receptor agents that inhibit the binding of HIV-1 to CXCR4 and CCR5.

Synthetic Peptides

Exemplary peptides 15D, 15K and 15KS, were synthesized using known methods by Macromolecular Resources (Fort Collins, Colo.), peptide 15CW was synthesized by the Basic Research Laboratory National Cancer Institute, (Frederick, MD). The peptides were purified by reverse-phase HPLC and the homogeneity of the peptide preparations was confirmed by mass-spectrometry.

Computer Modeling

Computer-generated structural models were derived using non-hydrogen atom superimposition of homologous residues during an optimization protocol of constrained consistent valence force field (CVFF) (Dauber-Osguthorpe et al., Proteins 4:31-47, 1988; Hagler et al., J. Am. Chem. Soc. 96:5319-27, 1974; and Hagler et al., Science 227:1309-15, 1985, each incorporated herein by reference), molecular dynamics sampling and conjugate gradients minimization of sampled structures.

Cells and Culture Conditions

HEK-293 cells expressing human CCR5 (HEK-CCR5) and CXCR4 (HEK-CXCR4) (Howard et al., J. Biol. Chem. 274:16228-16234, 1999, incorporated herein by reference) were cultured in Dulbecco's modified Eagle's medium (BioWhittaker, Walkersville, Md.) containing 10% fetal bovine serum (HyClone, Logan, Utah), 2 mM glutamine, 100 units/ml penicillin and streptomycin (Quality Biologicals, Gaithersburg, Md.) and 400 mg/ml Geneticin (Life Technologies, Inc., Rockville, Md.) at 37° C. in a humidified 5% CO₂ atmosphere. CEMx174 and THP-1 cell lines were obtained from ATCC (Rockville, Md.). Sup-T1 cells expressing CCR5 was the gift of James Hoxie. Cells were cultured in RPMI-1640 medium (BioWhittaker, Walkersville, Md.) containing 10% fetal bovine serum (FBS; HyClone, Logan, Utah), 2 mM glutamine, 100 units/ml penicillin and streptomycin (Quality Biologicals, Gaithersburg, Md.) at 37° C. in a humidified 5% CO₂ atrnosphere.

Binding Assays

Binding assays were performed with HEK-CCR5 or HEK CXCR4 in triplicate by adding unlabeled competitor (anti-coreceptor binding peptide or control chemokine) and radiolabeled chemokine, 0.2 ng/ml (¹²⁵I-MIP-1β or SDF-1α, specific activity 2000 Ci/mmol, NEN Life Science Products) to a cell suspension (4×10⁵ cells in 200 μl) in RPMI 1640 supplemented with 1% bovine serum albumin and 25 mM HEPES pH 8.0 (binding medium). Cells were then incubated at 22° C. for 40 min with continuous rotation. After incubation cells were transferred to the tubes containing 800 μl of 10% sucrose in PBS and harvested by centrifugation. The supernatant was aspirated and the cell-associated radioactivity was measured using a 1272 Wallac gamma counter. Binding assays were performed with the 174xCEM and Sup-T1 cells expressing CCR5 in duplicates by adding unlabeled competitor (peptide or control chemokine) and radiolabeled chemokine, 0.2 ng/ml (¹²⁵I-MIP-1β or ¹²⁵I-SDF-1α, specific activity 2000 Ci/mmol, NEN life Science Products) to 300 μl cell suspension (4×10⁶ cells/ml) in RPMI 1640 supplemented with 1% bovine serum albumin, 0.1% sodium azide and 25 mM HEPES pH 8.0 (binding medium). Cells were then incubated at 22° C. for 30 min with continuous rotation. After incubation cells were transferred to the tubes containing 800 μl of 10% sucrose in PBS and harvested by centrifugation, the supematant was aspirated and the cell-associated radioactivity was measured using a 1272 Wallac gamma counter. The data were analyzed by nonlinear regression using the computer program GraphPad Prizm 3.0.

Chemotaxis Assays

Chemotaxis assays for HEK-293 cells transfected with CCRS was performed as previously described (Howard et al., J. Biol. Chem. 274:16228-16234, 1999, incorporated herein by reference). Briefly, the chemokine RANTES, diluted in binding medium to 100 ng/ml, was placed in the lower wells of a microchemotaxis chamber. Polycarbonate membrane pretreated with rat tail collagen type 1 was placed over the chemokine solution. The cells (1×10⁶/ml) were suspended in binding medium along with the exemplary peptides 15D or 15K at designated concentrations and placed in the upper wells of the chamber. After incubation for 3 hr the membrane was removed, stained using a Diff-Quik kit (Trends Scientific, Kalamazoo, Mich.) and counted. The results are expressed as the “chemotaxis index”, which represents the ratio of the number of cells in high powered field in test versus control samples.

Flow Cytometric Analysis

CEMx174 cells were pelleted and resuspended at 10⁶ cells/ml in PBS containing 1% BSA and 0.1% sodium azide (FACS buffer). Cells were then preincubated with designated concentrations of peptides or SDF-1-α (1 μg/ml) at 22° C. for 60 min. FITC-labeled anti-human CXCR4 antibody (clone 12G5, BD PharMingen, San Diego, Calif.) was added to the cells per the manufactures instructions and further incubated at 22° C. for 40 minutes. Cells were extensively washed with FACS buffer and analyzed using a FACS Calibur flowcytometer (Becton Dickinson). HEK/CCR5 cells were suspended in Dulbecco's PBS containing 1% FCS and 0.05% NaN₃ (10⁴ cells in 100 μl) and incubated with or without 5 μg of MIP-1β (PeproTech, Rocky Hill, N.J.), 0.1 mM peptide 15D or 15K for 45 min at 22° C. Cells were then treated with FITC-conjugated anti-human CCR5 antibody (2D7; BD PharMingen, San Diego, Calif.), and incubated for 30 min at 22° C. Cells were washed twice, and analyzed using a FACScan flow cytometer (Becton Dickinson Immunocytometry Systems, San Jose, Calif.).

CA²⁺ Mobilization Assay

THP-1 cells (12×10⁶/ml in RPMI 1640 containing 10% FSB) were loaded in the presence of 5 μM fura-2 AM (Molecular Probes, Eugene, Oreg.) at 22° C. for 30 min in the dark. Subsequently, cells were washed three times and resuspended (10⁶ cells/ml) in the buffer containing 138 mM sodium chloride, 6 mM potassium chloride, 1 mM calcium chloride, 1 mM magnesium chloride, 10 mM HEPES, pH 7.4,5 mM glucose, and 0.1% bovine serum albumin. 1.93 ml of loaded cells was transferred into a quartz cuvette. 50 μl of a peptide stock solution (2×10⁻² M) was added to the cells, and after 3 min of incubation 20 μl of human SDF-1α (10 μg/ml, PeproTech) was added to a stirred cuvette. The measurements were performed using luminescence spectrophotometer LS50 B (Perkin-Elmer). Ca²⁺ mobilization in the cells in response to SDF-1α was measured by analysis of the ratio of fluorescence emitted at 510 nm after sequential excitation at 340 and 380 nm.

Assays of HIV Infectivity

PBMCs were obtained from whole blood of normal donors and isolated by Ficoll-Paque Plus (Pharmacia Biotech, Piscataway, N.J.) density gradient centrifugation, and plated at a cell density of 2×10⁶ cells/ml. Monocyte-derived macrophages (MDM) were generated from adherent human peripheral blood mononuclear cells by culture for 7 days with M-CSF (100 ng/ml). Cultures were maintained in RPMI-1640 medium (Life Technologies, Rockville, Md.) supplemented with 10% heat-inactivated endotoxin-free FCS (Hyclone, Logan, Utah), 10 μg/ml gentamicin, and 1 mM glutamine. Cells were treated with designated concentrations of the exemplary peptides 15K or 15D, and after 1 hr cells were infected with HIV-1_{IIIB} (T cell tropic) or HIV-1_{JRFL} (monocyte tropic) at an multiplicity of infection (MOI) of 0.1. After 2 hr, cells were washed, and cultured for additional 48-72 hr, followed by analysis of HIV replication as determined by quantification of accumulated p24 in the supernatant. The production of p24 was determined by conventional sandwich ELISA, using ELISA plates pre-coated with capture anti-p24 antibodies provided by the AIDS Vaccine Program (SAIC Frederick, NCI-FCRDC, Frederick, Md.). The captured p24 antigen was detected using rabbit anti-HIV-1 anti-p24 antibody, and a secondary goat anti-rabbit IgG (peroxidase-labeled) antibody. The captured p24 protein was detected using a 3,3′,5,5′-tetramethylbenzidine and hydrogen peroxide detection system (KPL Laboratories, Gaithersburg, Md.). The reaction was read spectrophotometrically at 450 nm. No cytotoxicity was detectable following treatment of PBMC with the peptides even at a concentration of 100 μg/ml.

### Example I

### Structural Analysis of gp120 and Chemokines

As noted above, a number of recent studies point to a role of chemokine receptors as coreceptors for HIV cell entry. Additional studies suggest that protein fragments or peptides from chemokines or HIV corresponding to structural determinants involved in chemokine receptor (coreceptor) binding, may be useful to block HIV-coreceptor binding and therefore serve as anti-HIV reagents. Considering that both chemokines and the HIV envelope protein gp120 are thought to directly interact with chemokine receptors, it is conceivable that this direct interaction may involve a part of the gp120 polypeptide chain that is structurally similar to (e.g., by homologous or convergent evolutionary relationship) receptor binding determinants of chemokines.

However, conventional amino acid sequence comparison between gp120 and chemokines undertaken by the present inventors did not reveal any significant amino acid identity or similarity relationship that would point to a convergent, or conservative, chemokine receptor binding domain. Despite this apparent lack of structural homology, the present investigation focused on a putative structural element found to be conserved within the amino acid sequences of nearly all chemokines, and also shared in a corresponding structural motif postulated for the HIV-1 gp120 protein. In particular, all chemokines studied were found to possess a Trp residue located at the beginning of a C-terminal alpha-helix. This conserved Trp residue is separated by six residues from a 4^{th} Cys residue, to comprise what is characterized herein as a conserved chemokine structural motif. By comparison, in the amino acid sequences of gp120 proteins of all HIV-1 isolates there is a very similar motif in a spanning part of the conserved region 3 (C3) adjacent to the V3 loop. Surprisingly, this motif was also found to include one or more residues within a C-terminal portion of the variable loop 3 (V3) of gp120 that are conserved between different HIV-1 strains and exhibit some homology with chemokines (Table 1).

### Example II

### Computer Modeling of gp120 and Chemokines

Computer modeling further demonstrates that novel peptide fragments of gp120 can be template forced onto a “homologous loop” of the known structure of an exemplary chemokine, MIP-1β without violation of the general rules of protein structure. To assess the possibility that a selected region of gp120 may potentially assume a structure similar to that of chemokines, a model of the corresponding structure of gp120 was built using the structure of MIP-1β (Lodi et al., Science 263:1762-1767, 1994, incorporated herein by reference) as the template. Based on this analysis, it was determined that a model of the three-dimensional structure of the selected gp120 segment could be generated without violation of protein stereochemistry. In an effort to examine the functional activity of this region, peptides corresponding to the selected gp120 sequence were synthesized for further studies.

### Example III

### Peptide Design and Synthesis

Two exemplary peptides were synthesized based on a reference peptide comprising a C-terminal portion of the V3 loop and an N-terminal of the C3 domain of gp120 (see Table 4, above). The sequence of HIV-1_{JRFL}, which is very close to the consensus B of HIV-1 sequences (Korber and Los Alamos National Laboratory-Theoretical Biology and Biophysics Group T-10, Human Retroviruses and AIDS, 1998, A Compilation and Analysis of Nucleic Acid and Amino Acid Sequences, Los Alamos National Laboratory, Los Alamos, NW, 1998), was used as a template for the design of anti-HIV peptides.

Synthesized peptides corresponding to a conserved structural motif of the HlV1_{JRFL} gp120 protein, bearing the above-noted structural similarity to a corresponding, conserved motif identified in chemokines, are shown in Table 4. Because gp120 of different HIV-1 strains have some sequences variabilities even in conserved regions, peptides were designed with amino acid sequences similar to the widest range of naturally existing variants. In many HIV strains there is a lysine (k) residue instead of glutamine (Q) in position 3 (Table 4). Moreover, it was reasonable to change glutamine for lysine in the synthetic peptide to avoid hydrolysis and conversion to glutamic acid near the conserved positively changed arginine. The lysine residue preceding the tryptophan (W) in the peptide designated 15K was changed to aspartic acid (D) in the peptides designated 15D because some HIV-1 isolates have aspartic acid in this position and the change provided an opportunity to explore the significance of this substitution. For control experiments, peptides with “scrambled” sequences were also synthesized (peptide 15CW with the same amino acid composition as 15D, and 15KS with the same amino acid composition as 15K). In some control experiments a 15 amino acid peptide, 15GIG, with an amino acid sequence unrelated to gp120 was also used (See Table 4).

TABLE 4
SEQUENCE OF SYNTHESIZED PEPTIDES

| | |
|---|---|
| | SEQ |
| Amino Acid Sequence | ID |

| | |
|---|---|
| 1 2 3 4 5 6 7 8 9 10 11 12 13 14 15 | NO: |

| | | |
|---|---|---|
| HIV_{JRFL} | I R Q A H C N I S R A K W N D | SEQ |
| | | ID |
| | | NO:3 |
| 15 K | I R K A H C N I S R A K W N D | SEQ |
| | | ID |
| | | NO:8 |
| 15 D | I R K A H C N I S R A D W N D | SEQ |
| | | ID |
| | | NO:9 |
| 15CW | I R K A H C W I D R A D N N S | SEQ |
| | | ID |
| | | NO:10 |
| 15KS | K I N S W R A D N I H C K A R | SEQ |
| | | ID |
| | | NO:11 |
| 15GIG | G I G D P V T C L K S G A I A | SEQ |
| | | ID |
| | | NO:12 |

As further detailed below, these exemplary peptides exhibited surprising anti-coreceptor binding activities, including competition with chemokines for binding to CCR5- and CXCR4-expressing cells, and inhibition of chemotaxis in chemokine-responsive cells. Correlated with these activities, the peptides mediate potent inhibition of HIV replication in macrophages and T lymphocytes, evincing their efficacy for prophylaxis and treatment of HIV infection and related disease conditions within the compositions and methods of the invention. This and related description herein further provides abundant structural information to enable the design and construction of a large assemblage of effective anti-coreceptor binding peptides, peptide analogs and mimetics, to serve as potent inhibitors of HIV-coreceptor interactions.

### Example IV

### Anti-Coreceptor Binding Activity of gp120 Peptides as Demonstrated by Inhibition of Chemokine Binding to CCR5 and CXCR4 Expressing Cells

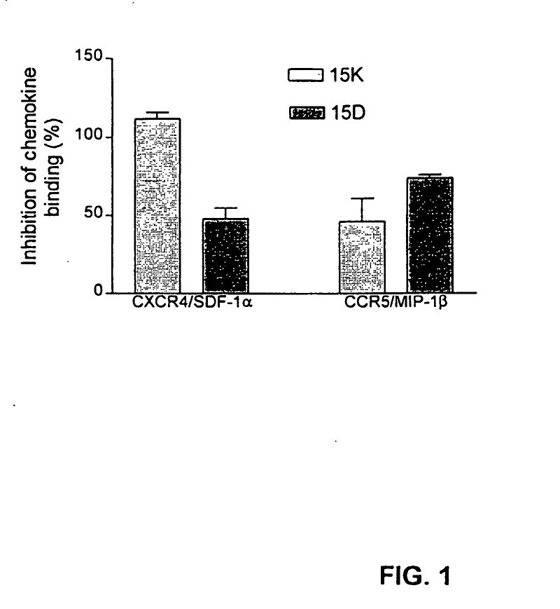

Considering that the 15K and 15D peptides were designed in part based on their structural similarity to corresponding chemokine fragments, the binding activity of these peptides for CCR5 and/or CXCR4 receptors, and corresponding anti-coreceptor binding activity against chemokines, was assessed. Experiments were carried out to determine the ability of these exemplary peptides to competitively inhibit binding of radiolabeled MIP-1β or SDF-1α to cells expressing CCR5 or CXCR4, respectively. The results, shown in FIG. 1, demonstrate that both peptides significantly inhibit chemokine binding, although high concentrations (e.g., 100 μM) of peptide are required to mediate this effect.

### Example V

### Anti-Coreceptor Binding Activity of gp120 Peptides as Demonstrated by Inhibition of CCR5-Mediated Chemotaxis

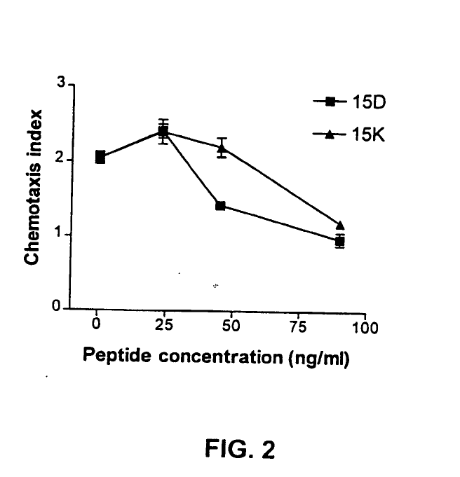

The inhibition of binding of MIP-1β and SDF-1α to their respective receptors suggests that the exemplary peptides 15K and 15D might also inhibit chemotaxis of cells expressing cognate chemokine receptors for appropriate ligands. To achieve this goal, the capacity of these peptides to inhibit the chemotactic response of HEK-CCR5 cells to a cognate ligand of CCR5 receptors, the chemokine RANTES, was assayed. The results demonstrated that RANTES-directed chemotactic responses were completely blocked by the addition of either 15K or 15D (FIG. 2). Interestingly, one of the peptides, 15D, consistently exhibited greater inhibitory activity in these experiments. These results show that the 15K and 15D peptides interfere with chemokine receptor function.

### Example VI

### Anti-Coreceptor Binding Activity of gp120 Peptides Mediates Inhibition of HIV Infection in Monocyte-Tropic Cells

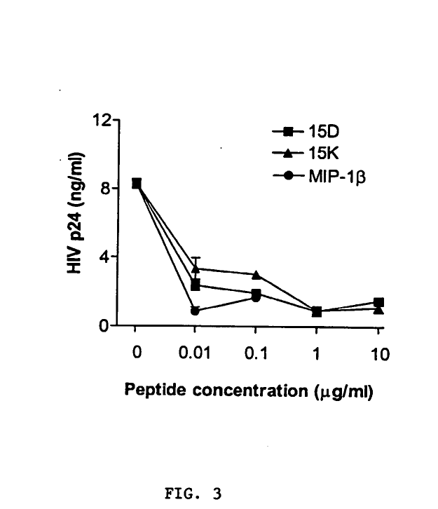

The results described in the preceding Examples indicate that the 15K and 15D peptides effectively block the ability of CCR5 to serve as a receptor to mediate chemotactic responses. Additional experiments were thus carried out to determine the capacity of these peptides to inhibit coreceptor activity mediating cellular entry and infection by HIV-1_{JRFL} (a monocyte-tropic HIV strain), using peripheral blood monocyte-derived macrophages. The results, presented in FIG. 3, show that the addition of either 15K or 15D significantly reduced monocyte lineage target cell infection by HIV-1. Significant inhibition was exhibited at concentrations as low as 10 ng/ml. Interestingly, the CCR5-selective chemokine ligand MIP-1β and the 15K and 15D peptides exhibited comparable inhibitory activities (FIG. 3).

### Example VII

### Anti-Coreceptor Binding Activity of gp120 Peptides Mediates Inhibition of HIV Infection in Lymphocyte-Tropic Cells

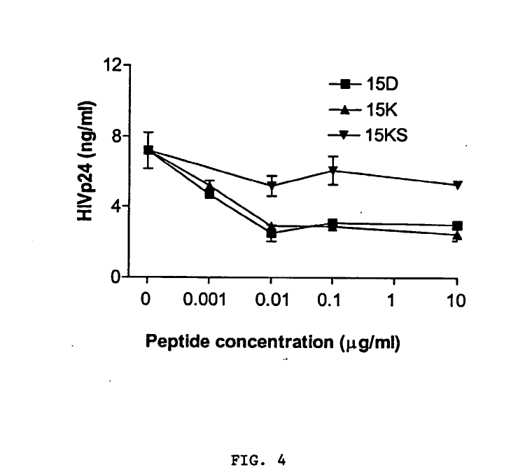

In view of the foregoing binding inhibition studies, and considering the homology between the 15K and 15D peptides and the CXCR4 ligand SDF-1α, it was further undertaken to determine the capacity of the synthetic peptides to inhibit CXCR4 co-receptor function. In particular, experiments were carried out to assess the ability of 15K or 15D to alter the infection of PBMCs with the T cell-tropic HIV-1β strain. The results, presented in FIG. 4, demonstrate that both peptides significantly reduced HIV-1 infection, in a dose-dependent manner. Significant inhibition of HIV-1 infection was detected with concentrations of peptides as low as 10 ng/ml. The scrambled peptide 15KS with the same amino acid composition as 15K but a randomized sequence manifested significantly less inhibition of virus infectively (FIG. 4) demonstrating the importance of the sequence of amino acids for inhibiting activity.

### Example VIII

### Activity of gp120 Peptides in Relation to Binding of Anti-Coreceptor Antibodies

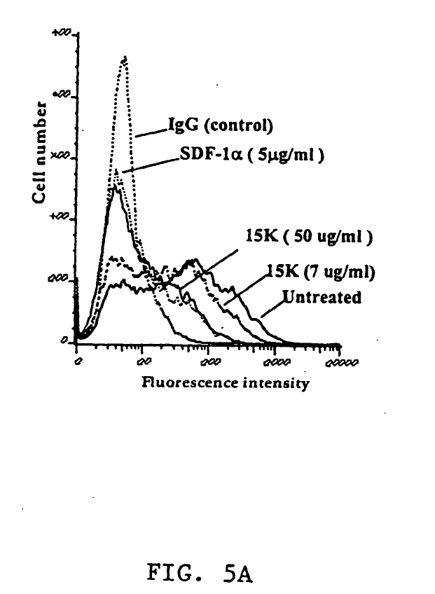

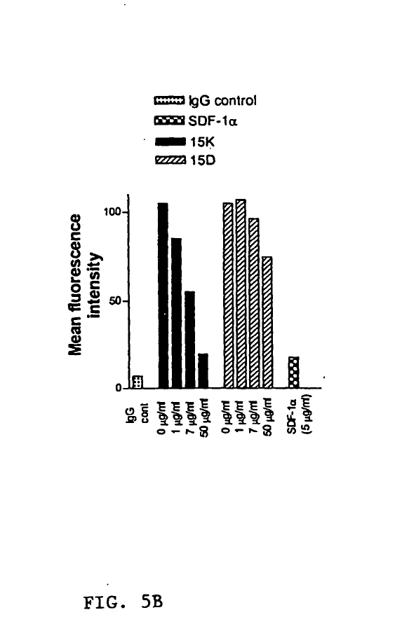

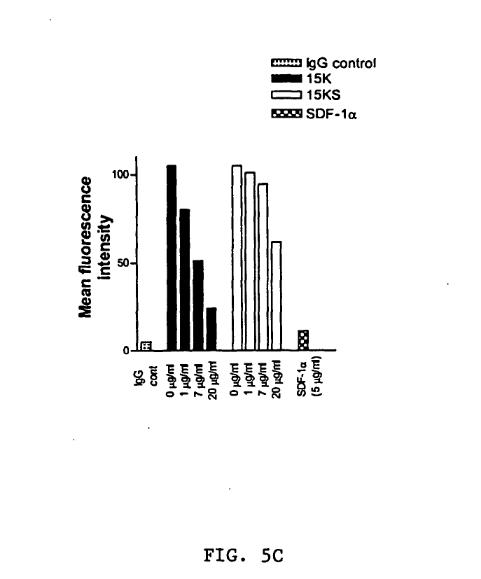

To determine if peptides 15D and 15K interact directly with chemokine receptors the effect of these and control peptides on the binding of anti-chemokine receptor antibodies to CXCR4 and CCR5 was studied. The monoclonal antibody 12G5 recognizes a conformational extracellular epitope on CXCR4. This antibody blocks the infectivity of some X4 strains of HIV-1 and HIV-2 (Endres et al., Cell 87:745-756, 1996; Hoxie et al., J. Reprod. Immunol. 41:197-211(1998); McKnight et al., J. Virol. 74:1692-1696(1997), 19, 26). It also inhibits the binding of SDF-1α, a natural CXCR4 ligand Schols et al., J. Exp. Med. 186:1383-1388, 1997 (Haste et al., Mol. Pharmacol. 60:164-173(2001), 33). Further, the binding of 12G5 to CXCR4 receptor-expressing cells is prevented by anti-HIV compounds. The results show that peptide 15K inhibited the binding of the 12G5 antibody to CXCR4 in a dose-dependent manner (FIG. 5A), and at a concentration of 50 μg/ml approached the efficacy of SDF-1α at 5 mg/ml. Peptide 15D appeared to be less effective in blocking of 12G5 antibody binding to CXCR4 (FIG. 5B), which is not surprising since the binding site of CXCR4 includes several negatively charged residues Duranz et al., J. Virol. 73:2752-2761, 1999. To determine the importance of the particular amino acid sequence of the inhibitory activity of the peptides. The effects of peptide 15K and the corresponding scrambled peptide 15KS on binding of 12G5 to CXCR4 was compared. The scrambled peptide 15KS manifest significantly less inhibitory activity in comparison with 15K suggesting the importance of the specific peptide sequence (FIG. 5C). Apparently there is a direct correlation between the ability of 15K to inhibit the anti-CXCR4 antibody binding and its ability to inhibit HIV-1 infectivity since the scrambled peptide 15KS with the same amino acid comparison has reduced ability to manifest both these activities.

The effect of the exemplary peptides 15K and 15D on the binding of 2D7 antibodies to CCR5 was assayed to further evaluate the mechanism of anti-coreceptor activity of the peptides. The 2D7 monoclonal antibody binds to a conformational epitope on CCR5, and inhibits Ca²⁺ flux induced by RANTES. This antibody has also been shown to inhibit HIV infection in vitro (Wu et al., J. Exp. Med. 186:1373-81, 1997, incorporated herein by reference). It does not, however, inhibit binding of gp120-SCD4 complexes to CCR5-expressing L1.2 murine cells (Olson et al., J. Virol. 73:4145-4155, 1999, incorporated herein by reference).

The effects of peptides 15D and 15K on 2D7 antibody binding to HEK293/CCR5 cells were analyzed by flow cytometry. No significant effect of either of the two peptides on anti-CCR5 antibody binding was observed. These results indicate that the peptides bind to an epitope on the CCR5 receptor that is spatially and sterically distinct from the antibody binding site. Considering that both the peptides and the 2D7 antibodies inhibit HIV infection in vitro, these results may be interpreted as underscoring the unexpected nature and advantages of the present invention.

### Example IX

### Effects of Peptides on Induction of Intracellular Ca²⁺ Concentration in CXCR4 Expressing THP-1 Cells

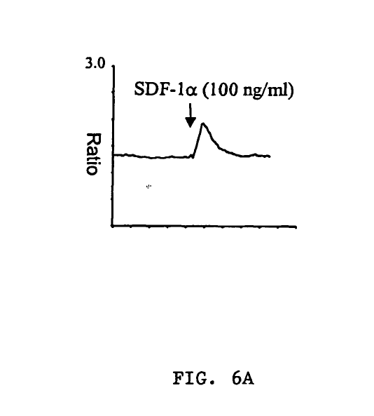

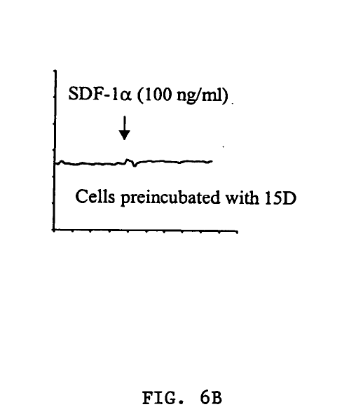

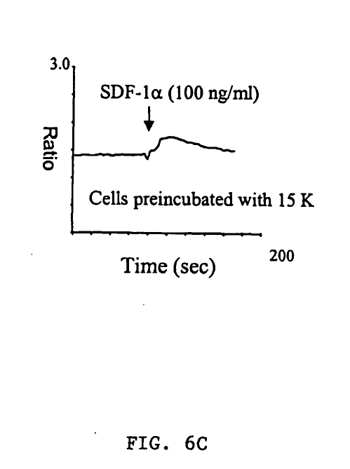

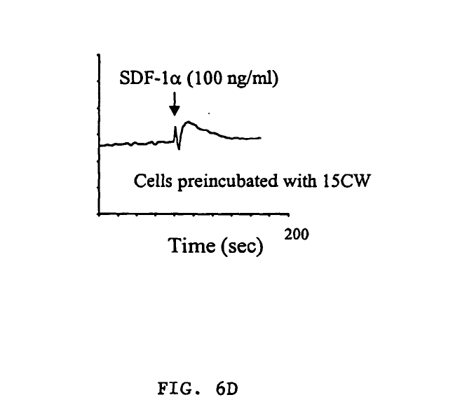

The increase of intracellular Ca²⁺ concentration mediated by a chemokine receptor in response to a cognate chemokine is a reliable assay for measurement of chemokine agonist activity. The human monocytic cell line THP-1 expresses a high level of CXCR4 and responds well to SDF-1α stimulation (FIG. 6A), so these cells were used to determine whether peptides 15D and 15K could block the activation of CXCR4. Preincubation of cells with peptide 15D (at a concentration 500 μM) for 2 min completely inhibited cellular response to SDF-1α (FIG. 6B). Peptide 15K also inhibited Ca²⁺ mobilization although less efficiently than 15D (FIG. 6C). There is a probable direct correlation between the greater ability of 15D to inhibit Ca²⁺ mobilization in response to SDF-1α and anti-HIV-1 activity because in some experiments higher anti-HIV-1 activity of 15D was observed in comparison with 15K (data not shown). The scrambled peptide 15CW, with amino acid composition identical to 15D (Table 4), was less effective at inhibiting the Ca²⁺ mobilization response (FIG. 6D) suggesting the importance of the specific peptide sequence. The peptide treatment alone did not induce Ca²⁺ mobilization.

### Example X

### Effect of gp120 Derived Peptides 15K and 15D on Binding of Chemokines to CCRS and CXCR4 Expressing Cells

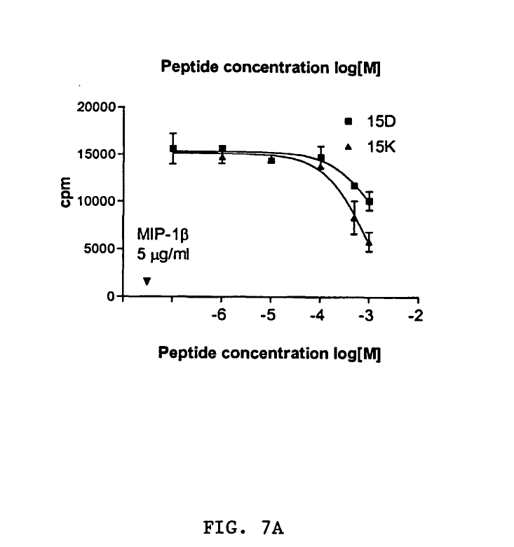

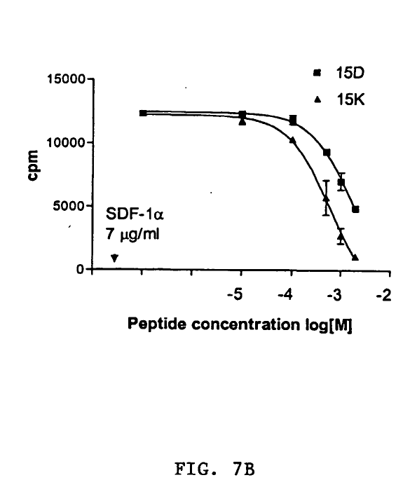

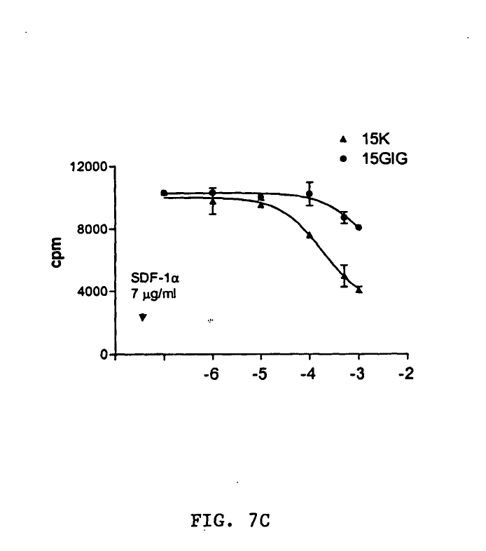

Since the 15K and 15D peptides were designed based on their structural homology with a chemokine fragment, whether these peptides might exhibit binding activity for CCR5 or CXCR4 was determined. Experiments were carried out to assess the ability of the peptides to competitively inhibit binding of radiolabeled MIP-1β or SDF-1α to cells expressing CCR4 or CXCR4, respectively. To determine the effect of peptides exclusively on chemokine binding and to exclude the contribution of chemokine internalization, cells were incubated at room temperature in the presence of 0.1% sodium azide. Under such conditions all cell-bound ligand could be removed by rinsing the cells with 0.1 M Gly-HCl pH 2.5, excluding the contribution of internalization to the chemokine binding. The binding experiments demonstrated that the 15D and 15K peptides competitively inhibited chemokine binding to CCR5 (FIG. 7A) and CXCR4 (FIG. 7B). The binding experiments did not reveal any difference in the inhibitory activity of peptides 15K and 15D in comparison with their scrambled analogs (data not shown). The control 15-mer peptide 15GIG with a different amino acid composition manifested significantly lower competing activity (FIG. 7C). It is possible that the particular scrambling of the peptide sequence that was performed did not change the sequences of the scrambled peptides sufficiently to significantly reduce their capacity to bind to the receptors because scrambling of these peptides may have created similar amino acid triplets in other positions of the peptides (like RAK and KAR in 15K and 15KS, Table 4). It is possible that because of this peptide 15KS also manifested a low level of inhibitory activity (although significantly less than original peptide) on anti-CXCR4 antibody binding to the receptor (FIG. 5C). Additional structure-functional studies are necessary to determine the minimum sequence of the peptides sufficient for inhibition of virus.

To briefly summarize the foregoing description, the interaction of HIV envelope glycoprotein gp120 with a chemokine receptor is known to be a prerequisite for viral attachment and entry into target cells (Alkhatib et al., Science 272:1955-1958, 1996; Berson et al., J. Virol. 70:6288-6295, 1996; Deng et al., Nature 381:661-666, 1996; and Dragic et al., Nature 381:667-673, 1996, each incorporated herein by reference). Monocyte-tropic viruses (R5 strains) utilize a distinct chemokine receptor, CCR5, for cell entry, while T cell tropic viruses (X4 strains) utilize CXCR4 receptors. Chemokine receptor binding site(s) on gp120 are thought to be formed, or exposed, after binding of gp120 to CD4 (Wu et al., Nature 384:179-183, 1996, incorporated herein by reference). However, gp120 binding to the chemokine receptor in certain HIV strains does not require interaction with CD4 (Hoffman et al., Proc. Natl. Acad. Sci. USA 96:6359-6364, 1999; and Hoxie et al., J. Reprod. Immunol. 41:197-211, 1998, each incorporated herein by reference).

The V3 loop of gp120 has been identified as the major determinant of cellular tropism and coreceptor specificity (Cocchi et al., Nat. Med. 2:1244-1247, 1996; and Hwang, et al., Science 253:71-74, 1991, each incorporated herein by reference). Nevertheless, the precise region or residues within this 35-37 amino acid V3 loop responsible for mediating these phenotypic effects has not yet been established (Hung et al., J. Virol. 73:8216-8226, 1999, incorporated herein by reference). Synthetic cyclized peptides corresponding to the V3 loop of gp120 of X4 and dual strains of HIV-1 (but not an R5 strain) at micromolar concentrations could prevent binding of anti-CXCR4 antibodies. Some of these peptides at micromolar concentrations inhibited the infectivity of HIV-IIIB (Sakaida et al., J. Virol. 72:9763-9770, 1998, incorporated herein by reference). Synthetic polymer preparations including a putative V3 consensus sequence (GPGRAF, SEQ ID NO:13) of HIV-1 were reported to inhibit HIV-1 infection by an unknown mechanism. However, it is unlikely that this inhibition was due to competition with gp120 binding to the chemokine receptor, or to CD4 (Moulard et al., J. Pept. Res. 53:647-655, 1999, incorporated herein by reference). Although the influence of V3 on Hw coreceptor utilization is well established, other more conservative regions of gp120 are also proposed to be involved in coreceptor usage (Rizzuto et al., Science 280:1949-1953, 1998, incorporated herein by reference).

Within the present invention, a conservative chemokine receptor binding motif of gp120 was identified which shares certain similarity in amino acid sequence that appears conserved among relevant chemokines. More specifically, the present inventors observed that in the amino acid sequences of most chemokines there is a conserved Trp residue separated by six amino acid residues from a fourth Cys residue. In comparison, the gp120 of all HIV isolates analyzed exhibited a comparable structural motif in the C3 region following the V3 loop. Moreover, several residues of the C-terminal part of V3 loop also manifest some homology with a canonical chemokine sequence (Table 1). Computer modeling as provided herein further demonstrated that the corresponding fragment of gp120 could be template forced onto a “homologous” loop of the known three dimensional structure of an exemplary chemokine MIP-1β (for specific MIP-1β structural detail, see, e.g., Lodi et al., Science 263:1762-7, 1994, incorporated herein by reference) without violation of protein stereochemistry.

Prompted in part by these discoveries, two candidate peptides were synthesized which exemplify the proposed gp120 structural motif (Table 2). Both candidate peptides were demonstrated to compete with chemokines for binding to CCR5- and CXCR4-expressing cells. In addition, the exemplary peptides each inhibited CCR5-directed chemotaxis. Correlated with these anti-coreceptor activities, the peptides mediated potent inhibit replication of both monocyte-tropic HIV-1_{JRFL} and T cell tropic HIV-1_{IIIB}.

The X-ray structure of an HIV-1 gp120 core (not including V-loops), complexed with a two-domain fragment of CD4, and an antigen-binding fragment of neutralizing antibody, has been reported (Kwong et al., Nature 393:648-659, 1998). Although the structure does not include variable loops of gp120 it did include the C3 portion of the fragment of gp120, which the present invention considered to be structurally similar to the chemokines and potentially involved in interaction with chemokine receptors. In fact, the three-dimensional structure of this fragment (HIV_{HXB2}, residues 331-340, Table 1) appeared to be similar to the corresponding MIP-1β fragment. Both motifs are characterized by an α-helix preceded by a turn. Both contain a Trp residue with the indole ring buried in the interior region of the turn and with the α-carbon present on an exterior turn of the helix. The surface residues on the helices are characterized by long aliphatic side chains, terminated with polar groups, including Lys, Glu, Gln, and Asn. Importantly, this fragment is located within the region of gp120 molecule, which was implicated in CCR5 binding (Rizzuto et al., Science 280:1949-1953, 1998).

Since the peptides 15D and 15K include only 5 residues from the C-terminal part of the V3 loop, which is more conserved than other parts of V3 loop, and 9 residues from the N-terminal part of the conservative C3 region, it was reasonable to expect that anti-HIV activity of these peptides may not be dependent on virus tropism. The effects of the peptides on HIV-1 infectivity using R5 and X4 viruses confirmed this prediction (FIGS. 3 and 4).

The data above demonstrating that peptides 15K and 15D compete with anti-CXCR4 antibody 12G5 suggests a direct interaction of the peptide with this receptor. Although the 15D peptide had reduced ability to block binding of the 12G5 antibody, the infectivity data indicates that the low affinity interaction of 15D was still sufficient to interfere with viral infection. Moreover, the inhibition of the SDF-1α induced intracellular Ca²⁺ influx by peptides 15D and 15K also supports the conclusion that both antiviral peptides interact with chemokine receptors.

The affinity of interaction of the peptides 15D and 15K with chemokine receptors is apparently low, as evidenced by the high concentration of the peptides required to inhibit anti-CXCR4 antibody binding, mobilization of intracellular Ca2+ in response to SDF-1α, and chemokine binding. However, even this low affinity interaction is sufficient for interference with HIV-1 infection based on the low concentration of peptides required for blocking of the viral infection of macrophages and T lymphocytes. Indeed, it is not necessary for an efficient inhibitor of virus interaction with the coreceptor to also be a strong competitor of chemokines binding to the same receptor, because the envelope protein of HIV-1 only mimicks the highly specific binding of a chemokine with its receptor and the affinity of this interaction can be quite low (Hoffman et al., Proc. Natl. Acad. Sci. USA 99:11215-11220, 2000). The use of low affinity anti-viral drugs interfering with HIV-1 coreceptor interaction may allow targeting multiple cellular receptors while maintaining the ability to inhibit interactions of a viral glycoprotein, which is subject to frequent mutation. This may provide the basis for the capacity of the present peptides to inhibit infection by viruses using different coreceptors. Moreover, the rather weak competition of peptides 15D and 15K with chemokines for receptor binding, together with potent inhibition of HIV-1 infectivity, can be therapeutically preferable to high affinity inhibitors of CXCR4 and CCR5, because 15K and 15D peptides would not compromise functions of potentially critical chemokines such as SDF-1α or the CCR5 ligands.

The present invention has identified a region of HIV-1 gp120, which is structurally similar to chemokines, and appears to be directly involved in the interaction with certain chemokine receptors. The above findings that these peptides inhibit HIV-1 infection of human monocyte-derived macrophages and T-lymphocytes at low nanomolar concentrations suggest that these peptides, their analogs, and peptide menetics, can be used to dissect gp120 interactions with different chemokine receptors and could serve as not only leads for the design of new peptide inhibitors of HIV-1 not restricted by viral tropism, but are useful themselves as therapeutic agents to prevent binding of HIV-1 to a susceptible cell thereby reducing infection and viral replication in the treatment and prevention of HIV infection and related disease. Moreover, it may also be possible that antibodies raised against this sequence of HIV-1 gp120 can also have anti-HIV protective and therapeutic activity by reducing or preventing HIV binding to a susceptible cell.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. All publications, patents, and patent applications cited herein are hereby incorporated by reference in their entirety for all purposes.

What is claimed is:

1. A composition comprising an effective amount of an anti-coreceptor binding agent to inhibit binding of a CXCR4 and/or CCR5 coreceptor of a subject by an HIV virus or viral protein, wherein the anti-coreceptor binding agent is a gp120 peptide, peptide analog or mimetic that specifically binds the CXCR4 and/or CCR5 coreceptor.

2. The composition of claim 1, wherein the gp120 peptide, peptide analog or mimetic is between about 12 and about 24 amino acid residues in length and comprises a conserved CXXXXXXW amino acid sequence motif, wherein X is any naturally occurring or synthetic amino acid or amino acid analog.

3. The composition of claim 1, wherein the peptide, peptide analog or mimetic is modified by addition, admixture, or conjugation of additional amino acids, peptides, proteins, chemical reagents or moieties which do not substantially alter the anti-coreceptor binding activity of the peptide.

4. The composition of claim 1, wherein the anti-coreceptor binding agent is a peptide comprising an allelic variant among native HIV gp120 peptide sequences.

5. The composition of claim 1, wherein the anti-coreceptor binding agent is formulated for delivery to subject selected from an isolated or bound coreceptor, a membrane or cell preparation comprising the coreceptor, a cell population, tissue or organ expressing the coreceptor, or a mammalian patient.

6. The composition of claim 5, wherein the anti-coreceptor binding agent is combined with a pharmaceutically acceptable carrier, diluent, excipient, or adjuvant for administration in a prophylactic or therapeutic effective dose to a mammalian patient to prevent or inhibit HIV infection or a related disease condition or symptom in the patient.

7. The composition of claim 1, wherein the anti-coreceptor binding inhibits one or more biological activities mediated by or associated with HIV-coreceptor interactions selected from (a) direct co-receptor binding by HIV virus, (b) coreceptor binding by a HIV gp120 protein or a peptide fragment or derivative thereof, (c) HIV fusion with target host cells, (d) HIV virion entry into host cells, (e) HIV replication, and/or (f) HIV cell-cell or host-host transmission.

8. The composition of claim 1, wherein the anti-coreceptor binding agent comprises an effective formulation of an HIV-1 peptide, peptide analog or mimetic for in vivo administration to inhibit one or more biological activities selected from (a) direct co-receptor binding by HIV-1 virus, (b) coreceptor binding by a HIV-1 gp120 protein or a peptide fragment or derivative thereof, (c) HIV-1 fusion with target host cells, (d) HIV-1 virion entry into host cells, (e) HIV-1 replication, and/or (f) HIV-1 cell-cell or host-host transmission.

9. The composition of claim 1, wherein the anti-coreceptor binding agent is an HIV-1 peptide, peptide analog or mimetic formulated for administration to a mammalian patient in a prophylactically or therapeutically effective dose to prevent or inhibit HIV-1 infection or an HIV-1-related disease condition or symptom.

10. The composition of claim 1, wherein the anti-coreceptor binding agent is a peptide that includes a conserved “CXXXXXXW” amino acid sequence motif, wherein X is any amino acid, and wherein the peptide is from about 12-17 amino acids in length and is selected from peptide 15K, comprising an amino acid sequence IRKAHCNISRAKWND (SEQ ID NO:8), or a corresponding or overlapping native peptide sequence or peptide analog that shares substantial sequence identity to the reference peptide sequence of 15K.

11. The composition of claim 10, wherein the peptide includes one or more residues occurring naturally or by substitution at a relative, aligned position corresponding to a designated position for peptide 15K, selected from:
Position 1—I, M, K, S, T, L, A, V, R, P, or N;
Position 2—R, G, E, K, S, T, or I;
Position 3—Q, K, R, L, E, P, A, V, S, T, H, or D;
Position 4—A, T, P, V, E, or S;
Position 5—H, Y, F, Q, N, I, or V;
Position 7—N, D, H, T, K, E, S, I, Q, V, G, or A;
Position 8—I, L, V, Y, D, A;
Position 9—S, N, D, T, K, Y, I, or P;
Position 10—R, K, G, S, A, E, D, I, T, W, or N;
Position 11—A, R, K, T, S, G, E, D, N, Q, H, V, I, or L;
Position 12—K, D, R, E, K, Q, N, T, S, G, A, V, L;
Position 14—N, Q, D, E, K, R, A, S, T, G, M, Y, I, H, or V; and/or
Position 15—D, N, K, E, T, Q, R, S, A, I, M, or P.

12. The composition of claim 1, wherein the anti-coreceptor binding agent exhibits multi-tropic activity characterized by effective inhibition of HIV viral, or gp120 protein or peptide binding to multiple, CXCR4 and CCR5, coreceptors.

13. The composition of claim 12, wherein the multi-tropic anti-coreceptor binding agent is an HIV-1 peptide, peptide analog or mimetic effective to inhibit one or more biological activities of both T cell tropic (lymphotropic) and macrophage tropic (m-tropic) HIV-1 viruses selected from (a) direct co-receptor binding by viruses, (b) coreceptor binding by viral gp120 proteins or peptide fragments or derivatives thereof, (c) viral fusion with target host cells, (d) virion entry into host cells, (e) viral replication, and/or (f) viral cell-cell or host-host transmission.

14. A method for inhibiting human immunodeficiency virus (HIV) interaction with a CXCR4 and/or CCR5 coreceptor comprising exposing a subject to an effective amount of an anti-coreceptor binding agent to inhibit binding of the CXCR4 and/or CCR5 coreceptor by an HIV virus or viral protein, wherein the anti-coreceptor binding agent is a gp120 peptide, peptide analog or mimetic that specifically binds the CXCR4 and/or CCR5 coreceptor.

15. The method of claim 14, wherein the gp120 peptide, peptide analog or mimetic is between about 12 and about 24 amino acid residues in length and comprises a conserved CXXXXXXW amino acid sequence motif, wherein X is any naturally occurring or synthetic amino acid or amino acid analog.

16. The method of claim 14, wherein the peptide, peptide analog or mimetic is modified by addition, admixture, or conjugation of additional amino acids, peptides, proteins, chemical reagents or moieties which do not substantially alter the anti-coreceptor binding activity of the peptide.

17. The method of claim 14, wherein the anti-coreceptor binding agent is a peptide comprising an allelic variant among native HIV gp120 peptide sequences.

18. The method of claim 14, wherein the subject is an isolated or bound CXCR4 and/or CCRS coreceptor, a membrane or cell preparation comprising the coreceptor, a cell population, tissue or organ expressing the coreceptor, or a mammalian patient.

19. The method of claim 18, wherein the subject comprises a cell population, tissue or organ selected for in vivo or ex vivo treatment or diagnostic processing.

20. The method of claim 18, wherein the subject is a mammalian patient susceptible to HIV infection and the anti-coreceptor binding agent is administered in a prophylactic or therapeutic effective dose to prevent or inhibit HIV infection or a related disease condition or symptom.

21. The method of claim 14, wherein the anti-coreceptor binding agent is administered to the subject in an amount effective to inhibit one or more biological activities mediated by or associated with HIV-coreceptor interactions selected from (a) direct co-receptor binding by HIV virus, (b) coreceptor binding by a HIV gp120 protein or a peptide fragment or derivative thereof, (c) HIV fusion with target host cells, (d) HIV virion entry into host cells, (e) HIV replication, and/or (f) HIV cell-cell or host-host transmission.

22. The method of claim 14, wherein the anti-coreceptor binding agent is an HIV-1 peptide, peptide analog or mimetic administered to the subject in an amount effective to inhibit one or more biological activities selected from (a) direct co-receptor binding by HIV-1 virus, (b) coreceptor binding by a HIV-1 gp120 protein or a peptide fragment or derivative thereof, (c) HIV-1 fusion with target host cells, (d) HIV-1 virion entry into host cells, (e) HIV-1 replication, and/or (f) HIV-1 cell-cell or host-host transmission.

23. The method of claim 14, wherein the anti-coreceptor binding agent is an HIV-1 peptide, peptide analog or mimetic administered to a mammalian patient in a prophylactically or therapeutically effective dose to prevent or inhibit HIV-1 infection or an HIV-1-related disease condition or symptom.

24. The method of claim 14, wherein the anti-coreceptor binding agent is a peptide that includes a conserved “CXXXXXXW” amino acid sequence motif, wherein X is any amino acid, and wherein the peptide is from about 12 to about 17 amino acids in length and is selected from peptide 15K, comprising an amino acid sequence IRKAHCNISRAKWND (SEQ ID NO:8), or a corresponding or overlapping native peptide sequence or peptide analog that shares substantial sequence identity to the peptide sequence of 15K.

25. The method of claim 24, wherein the peptide includes one or more residues occurring naturally or by substitution at a relative, aligned position corresponding to a designated position for peptide 15K, selected from:
Position 1—I, M, K, S, T, L, A, V, R, P, or N;
Position 2—R, G, E, K, S, T, or I;
Position 3—Q, K, R, L, E, P, A, V, S, T, H, or D;
Position 4—A, T, P, V, E, or S;
Position 5—H, Y, F, Q, N, I or V;
Position 7—N, D, H, T, K, E, S, I, Q, V, G, or A;
Position 8—I, L, Y, D, A;
Position 9—S, N, D, T, K, Y, I, or P;
Position 10—R, K, G, S, A, E, D, I, T, W, or N;
Position 11—A,R,K,T, S, G, E, D,N, Q, H, V, I, or L;
Position 12—K, D, R, E, K, Q, N, T, S, G, A, V, L;
Position 14—N, Q, D, E, K, R, A, S, T, G, M, Y, I, H, or V; and/or
Position 15—D, N, K, E, T, Q, R, S, A, I, M, or P.

26. The method of claim 14, wherein the anti-coreceptor binding agent exhibits multi-tropic activity characterized by effective inhibition of HIV viral, or gp120 protein or peptide binding to multiple, CXCR4 and CCR5, coreceptors.

27. The method of claim 26, wherein the multi-tropic anti-coreceptor binding agent is an HIV-1 peptide, peptide analog or mimetic administered to the subject in an amount effective to inhibit one or more biological activities of both T cell tropic (lymphotropic) and macrophage tropic (m-tropic) HIV-1 viruses selected from (a) direct co-receptor binding by viruses, (b) coreceptor binding by viral gp120 proteins or peptide fragments or derivatives thereof, (c) viral fusion with target host cells, (d) virion entry into host cells, (e) viral replication, and/or (f) viral cell-cell or host-host transmission.
